(19) United States
(12) Patent Application Publication
(10) Pub. No.: US 2019/0272725 A1
(43) Pub. Date: Sep. 5, 2019

(54) PHARMACOVIGILANCE SYSTEMS AND METHODS

(71) Applicant: New Sun Technologies, Inc., Sunnyvale, CA (US)

(72) Inventors: Sophia Viklund, Los Altos Hills, CA (US); Clark Snowdall, Boulder Creek, CA (US); Adrian Kaehler, Los Altos Hills, CA (US); Andrew Spix, Santa Cruz, CA (US)

(73) Assignee: New Sun Technologies, Inc., Sunnyvale, CA (US)

(21) Appl. No.: 16/418,069

(22) Filed: May 21, 2019

Related U.S. Application Data
(63) Continuation of application No. PCT/US2018/054028, filed on Oct. 2, 2018.
Application No. PCT/US2018/054028 is a continuation-in-part of application No. PCT/US2018/018426, filed on Feb. 15, 2018.
(60) Provisional application No. 62/566,935, filed on Oct. 2, 2017.
(60) Provisional application No. 62/629,697, filed on Feb. 13, 2018.
(60) Provisional application No. 62/685,872, filed on Jun. 15, 2018.
(60) Provisional application No. 62/738,390, filed on Sep. 28, 2018.
(60) Provisional application No. 62/459,519, filed on Feb. 15, 2017.
(60) Provisional application No. 62/480,537, filed on Apr. 3, 2017.
(60) Provisional application No. 62/553,845, filed on Sep. 2, 2017.
(60) Provisional application No. 62/566,935, filed on Oct. 2, 2017.
(60) Provisional application No. 62/629,697, filed on Feb. 13, 2018.

(51) Int. Cl. G08B 21/04; H04W 4/14; H04W 4/90; H04W 4/029; G16H 10/60; G16H 40/67; G16H 20/10; G06N 20/00; H04W 12/00; A61B 5/11; A61B 5/00; A61B 5/117

(52) U.S. Cl. CPC G08B 21/0423; A61B 5/1117; H04W 4/90; G08B 21/0461; H04W 4/029; G16H 10/60; G16H 40/67; G16H 20/10; G06N 20/00; H04W 12/00514; A61B 5/1118; A61B 5/746; A61B 5/1112; A61B 5/4833; A61B 5/6898; A61B 5/742; A61B 5/7267; A61B 5/117; A61B 5/7465; A61B 2562/0219; H04W 4/14

(57) ABSTRACT

A system for monitoring people's activity and/or health while at the same time preserving their privacy. Monitoring is accomplished by detecting state of people's electronic devices, e.g., a smart phone. When use of the monitored device deviates from an expected use an alert is automatically sent to a monitoring device. The relationships between users are optionally maintained in a social network. Privacy is strictly protected by reporting only deviations from use according to rules set by the person being monitored. Optionally an activity monitoring system receives data from multiple sources such as a home security system, IoT devices and a smartphone. The use of multiple data sources provides an improved activity monitoring system capable of distinguishing normal activity from abnormal activity that may be indicative of a physical or mental health condition. Variations in the monitored activity are used to identify potential health issues for the user. If a health issue is identified, an alert may be sent to a remote third party. A human augmented positive feedback loop is used to generate data for training of machine learning systems.

### CROSS-REFERENCE TO RELATED APPLICATIONS

This application is timely filed as a continuation application based upon PCT/US 18/054018 filed Oct. 2, 2018 (the '018 Application), a continuation in part of PCT/US18/018426 filed Feb. 15, 2018 (the '426 Application). The '426 Application claimed priority to U.S. provisional patent applications Ser. Nos. 621459,519 filed Feb. 15, 2017, 62/480,537 filed Apr. 3, 2017, 62/553,845 filed Sep. 2, 2017, 62/566,935 filed Oct. 2, 2017, and 62/629,697 filed Feb. 15, 2018. The '018 Application claimed priority to U.S. provisional patent applications Ser. Nos. 62/459,519 filed Feb. 15, 2017, 62/480,537 filed Apr. 3, 2017, 62/553,845 filed Sep. 2, 2017, 62/566,935 filed Oct. 2, 2017, 62/629,697 filed Feb. 15, 2018, 62/685,872 filed Jun. 15, 2018, and 62/738,390 filed Sep. 28, 2018. The disclosures of all the above patent applications are hereby incorporated herein by reference.

### BACKGROUND

### Field of the Invention

The invention is in the field of user activity monitoring, and in some embodiments monitoring for detection of medical conditions.

### Related Art

Medical alert devices have existed for several years. These devices enable a user to push a button during a medical emergency in order to make a call for help. There are also mobile applications that report one user's activity to third parties. However, these applications are highly intrusive and report activity without enough regard to the user's privacy.

Home security systems include detection devices such as camera, optical motion sensors, window sensors and pressure sensors. Such systems are typically configured to detect intrusion. Health monitoring systems include wearable devices that allow a user to press a button to request help.

Pharmacovigilance is the pharmacological science relating to the collection, detection, assessment, monitoring, and prevention of adverse effects with pharmaceutical products. These products may include prescription and non-prescription drugs.

Surgery and other trauma, such has giving birth, an accident or surgery can have serious consequences and aftereffects. For example, the occurrence of infection after surgery or giving birth can lead to dangerous conditions, including death.

### SUMMARY

Various embodiments of the invention represent significant improvement over past monitoring systems. For example, mobile devices such as a smartphone, tablet computer, or wearable computer can be used to monitor activity of a user, by detecting use of the mobile device. The monitored activity can include movement, location, use of a device interface, or similar requisite hardware devices. If the monitored activity or lack thereof deviates from an expected use of the device, an alert is generated. For example, alerts may be generated in response to a lack of movement of a device over an extended period, which may be indicative of a problem. By reporting information indicative of a problem, rather than normal every-day expected use of a device, the user's privacy is greatly improved.

The alert may be sent to a healthcare organization, caregivers, family, and/or close friends in a passive social network, e-mail, and/or via a messaging application. Alerts may be sent automatically in response to the monitored activity. As such, the user is not required to explicitly (manually) send an alert. In addition to alerts, which are indicative of problems, a user may choose to send a regular “digest” of their activity to those that follow their activity. A digest typically includes a high-level summary of the user's activity. For example, a daily or weekly digest can include an indication that the user walked more than 2000 steps/day and got out of their house at least once. The digest is configured to provide an indication of the well-being of the user without having to include a detailed private log of their activities. The digests, thus, do not compromise the user's privacy unnecessarily.

The monitored activity can include discrete events such as a rapid deceleration, a fall or a visit to a hospital. The monitored activity can include activity patterns such as how long a person sleeps or how often a person leaves home. In some embodiments, expected activity patterns are determined using a machine-based analysis of historic activity patterns, e.g., using an artificial neural network or other machine learning system.

The monitored activity optionally includes monitoring the use of multiple devices. For example, if a user has a movement sensor, pressure sensor, wearable device, smartphone, tablet computer, desktop and home security system, then use of all these devices may be jointly considered in determining whether to send an alert. For example, a lack of activity on all these devices may indicate a problem and a need to send an alert. In contrast, if the tablet computer and desktop computer are not used for a predetermined period, but the smartphone is used and the home security detects movement within the user's home, then an alert may not be sent.

In some embodiments, a social network includes both a basic and enhanced connection between members. The basic connections are like those found in Facebook or LinkedIn and allow basic functionality such as text messaging and sharing of content. Enhanced connections allow a first network member to (passively) monitor activity of a second network member. The monitored activity is typically distinct from those activities found in a basic connection. For example, a first user may monitor the movement of a mobile device of the second user, and automatically receive an alert if there is a lack of movement for a predetermined time.

In some embodiments, a messaging application configured to automatically generate messages between parties. The messaging application can include any of the features found in Facebook Messenger, WhatsUp, and/or iMessage. In addition, enhanced features allow a first user to (passively) monitor activity of a second user. The monitored activity being distinct from those activities found in traditional text messaging (e.g., the manual sending, and automatic acknowledgement and receipt of messages). For example, a first user may monitor the movement of a mobile device of the second user, and automatically receive an alert if there is a lack of movement for a predetermined time.

In some embodiments, the activity of a user may also be monitored using movement detectors that are each configured to detect movement within their respective area of regard. Such sensors may be camera, radar, sonic or infrared based, for example. If movement is not detected by a set of movement detectors within a specified time period, then an alert is generated. Optionally a lack of movement within the areas of regard and lack of movement of a mobile device is required for the automatic generation of an alert.

In various embodiments, an activity monitoring system uses sensors, such as those found in security systems, personal electronic devices, medical devices and IoT (Internet of Things) devices, to detect activity of a user. An alert may be sent to third parties when the detected activity deviates from an expected activity. For example, the activity monitoring system is optionally configured to send an alert in response to a lack of or decrease in activity of the user. A deviation from expected activity can be indicative of a physical or mental health condition. The change in activity can be determined using a plurality of sensors. For example, a lack of activity may be concluded if no activity is detected at any of a set of motion sensors. These motion sensors are optionally configured to both monitor activity of a user and to detect intruders. Sensors of different types of devices may be used to draw conclusions about the activity of a user. For example, if a user is at a grocery store as determined by their smartphone GPS, then a lack of motion detected by their home security system would be considered normal. In contrast, if a user is home and normally gets up early, then a lack of motion detected by their home security system by 10 AM may be considered an unexpected deviation from normal activity. Likewise, if a user is active on a tablet computer, that fact that they are not using their smartphone or moving around their home is less relevant than if they were not active on their tablet computer.

The activity monitoring system does not require an affirmative action by a user in order to send an alert indicating that there may be a problem related to the user's health. To the contrary, an alert may be sent as a result of a lack of action by the user. This approach provides benefit to the user in situations wherein the user is unable to press a button or wherein a health-related issue develops gradually over time.

The activity monitoring system can receive input from a variety of sources. For example, a user's activity may be monitored using a home security system, a smartphone, an IoT (Internet of Things) thermostat, camera, radar, motion sensor, and/or a vehicle GPS system. The abilities to select different inputs and to apply activity analysis logic to data received from these different inputs are distinguishing features of some embodiments. Further, trained machine learning may be configured to use the received data in combination to detect deviations from a user's detected activity.

Sensors, such as those found in a smartphone, are used to measure the activity level of a patient. Deviations from expected activity result in automatically generated alerts that are then provided to medical care providers. The criteria for sending an alert are optionally selected based on past activity levels of the patient and/or the identity of a pharmaceutical to be consumed by the patient. The criteria for sending an alert are optionally selected based on a trauma experienced by a patient. For example, a patient having blood clots removed from a leg may need to walk some but not too much following the surgery. A patent having given birth or having had surgery should have a prescribed level of activity.

Machine learning systems are optionally used for several functions of the activity monitoring system. For example, machine learning systems may be used to interpret sensor data and determine a specific activity or level of activity therefrom; and/or machine learning systems may be used to determine an expected activity level for a user.

Various embodiments of the invention include a computing device comprising: a display (e.g., a touch screen) configured to present a user interface to a user; an I/O configured to communicate data from the computing device using at least one communication channel; activity logic configured to detect use of the mobile computing device, wherein the use comprises use of the user interface, use of the communication channel, movement of the computing device, or any other sensor connected to the mobile computing device; reporting logic configured to report the detected use of the mobile computing device to a remote destination; and a microprocessor configured to execute at least the activity logic. The computing device is optionally a mobile device.

Various embodiments of the invention include a monitoring system comprising: an I/O configured to communicate to and from remote clients; connection logic configured to establish a relationship between a first of the remote clients and a second of the remote clients in a social network; alert logic configured to provide an alert to the second of the remote clients, the alert being in response to data characterizing use of the first of the remote clients, the data characterizing use indicating deviant use of the first of the remote clients (e.g., use that deviates from expected use), the alert being sent to the second of the remote clients because of the relationship between the first and second of the remote clients; and a microprocessor configured to execute at least the alert logic.

Various embodiments of the invention include a method of monitoring a person's activity, the method comprising: determining an expected use of a mobile device, the expected use including location of the mobile device, movement of the mobile device or use of a peripheral connected to the mobile device; detecting use of the mobile device; comparing the detected use of the mobile device to the expected use of the mobile device; and sending an alert to a remote location, the alert indicating that the detected use of the mobile device has deviated from the expected use of the mobile device.

Various embodiments of the invention include a method of managing a social network, the method comprising: providing the social network to multiple members, the social network including a basic connection between members and an enhanced connection between members, the enhanced connection including automatic monitoring of use of a mobile device of a first user; detecting a use of the mobile device; determining that the detected use is outside of an expected (predetermined or learned) use pattern; and providing an alert to a second user that the detected use is outside of the predetermined or learned user pattern, the provision of the alert being based on an enhanced connection between the first user and the second user.

Various embodiments of the invention include a method of managing a social network, the method comprising: receiving data representing a social network including multiple members, the social network further including features that allow text messaging between the members, each of the members having a set of connections to one or more others of the members; and providing an upgrade opportunity to a first of the members, the upgrade opportunity including an ability to establish an enhanced connection between the first of the members and a second of the members, the enhanced connection including automatic monitoring of use of a mobile device of the second of the members and reporting of the automatic monitoring to the first of the members, wherein the monitored use is a use other than the messaging between the first and second members.

Various embodiments of the invention include an activity monitoring system comprising: a first motion sensor configured to detect movement within a first area of regard; a second motion sensor configured to detect movement within a second area of regard; activity logic configured to determine a lack of movement detected by both the first motion sensor and the second motion sensor for a first predetermined period of time; and reporting logic configured to report the lack of movement for the predetermined period of time to a remote destination.

Various embodiments of the invention include a method of monitoring a first person's activity, the method comprising: providing a first motion sensor having a first area of regard; providing a second motion sensor having a second area of regard; determining that motion has not been detected by either the first or second motion sensor for a predetermined period of time; and providing an alert to a remote destination, the alert indicating the lack of detected motion by either the first or second motion sensor.

Various embodiments of the invention include a pharmacovigilance system comprising: a sensor configured to detect movement or location; activity logic configured to determine an activity level of a patient based on the detected movement; threshold logic configured to set one or more criteria for generating an alert, based on an identity of a pharmaceutical prescribed to a patient; alert logic configured to generate an alert when the determined activity level satisfies the criteria for generating the alert; notice logic configured to send the alert to a caregiver of the patient; and a microprocessor configured to execute at least the threshold logic.

Various embodiments of the invention include a compliance system comprising: a sensor configured to detect movement or location; activity logic configured to determine an activity level of a patient based on the detected movement or location; threshold logic configured to set one or more criteria for generating an alert, based on an activity plan prescribed to a patient, the activity plan being based on a trauma experienced by the patient; alert logic configured to generate an alert when the determined activity level satisfies the criteria for generating the alert; notice logic configured to send the alert to a caregiver of the patient; and a microprocessor configured to execute at least the threshold logic.

Various embodiments of the invention include a method of monitoring a patient, the method comprising: characterizing a trauma experienced by a patient; selecting an activity plan for the patient based on the characterization of the trauma; receiving sensor data from a mobile device of the patient; using the sensor data to determine an activity level of the patient; determining that the determined activity satisfies criteria for generating an alert, the criteria being based on the activity plan; generating the alert; and sending the alert to a caregiver of the patient.

Various embodiments of the invention include a method of providing pharmacovigilance, the method comprising: obtaining a background activity level for a patient; determining one or more criteria for generating an alert, based on an identity and/or dosage of a pharmaceutical provided to the patient; receiving sensor data representative of movement of the patient; determining an activity level for the patient based on the sensor data; generating an alert responsive to the activity level meeting the criteria; and sending the alert to a medical provider of the patient.

### BRIEF DESCRIPTION OF THE DRAWINGS

FIG. 1 illustrates a block diagram of a passive monitoring network, according to various embodiments of the invention.

FIG. 2 illustrates a social network having more than one type of connection, according to various embodiments if the invention.

FIG. 3 illustrates methods of passively monitoring a user, according to various embodiments of the invention.

FIG. 4 illustrates methods of managing a social network, according to various embodiments of the invention.

FIG. 5 illustrates methods of upgrading a social network, according to various embodiments of the invention.

FIG. 6 illustrates methods of monitoring a person's activity, according to various embodiments of the invention.

FIG. 7 illustrates a sensor-based activity monitoring system, according to various embodiments of the invention.

FIG. 8 illustrates a device selection interface, according to various embodiments of the invention.

FIG. 9 illustrates methods of generating an alert, according to various embodiments of the invention.

FIG. 10 illustrates methods of training a machine learning system for a user, according to various embodiments of the invention.

FIG. 11 illustrates methods of generating an alert based on a dynamic threshold, according to various embodiments of the invention.

FIGS. 12A, 12B and 12C illustrate a real-time alert an alert cancellation interface and a digest, according to various embodiments of the invention.

FIG. 13 illustrates a method of determining a state of health, according to various embodiments of the invention.

FIG. 14 illustrates data flow between various elements of an activity monitoring system, according to various embodiments of the invention.

FIG. 15 illustrates data flow in personalized training of a machine learning system, according to various embodiments of the invention.

FIG. 16 illustrates methods of training a machine learning system, according to various embodiments of the invention.

FIG. 17 illustrates methods of personalized training of a machine learning system, according to various embodiments of the invention.

FIG. 18 illustrates a pharmacovigilance system, according to various embodiments of the invention.

FIG. 19 illustrates methods of providing pharmacovigilance, according to various embodiments of the invention.

FIG. 20 illustrates a patient's activity levels over time, according to various embodiments of the invention.

FIG. 21 illustrates a method of monitoring a patient, according to various embodiments of the invention.

### DETAILED DESCRIPTION

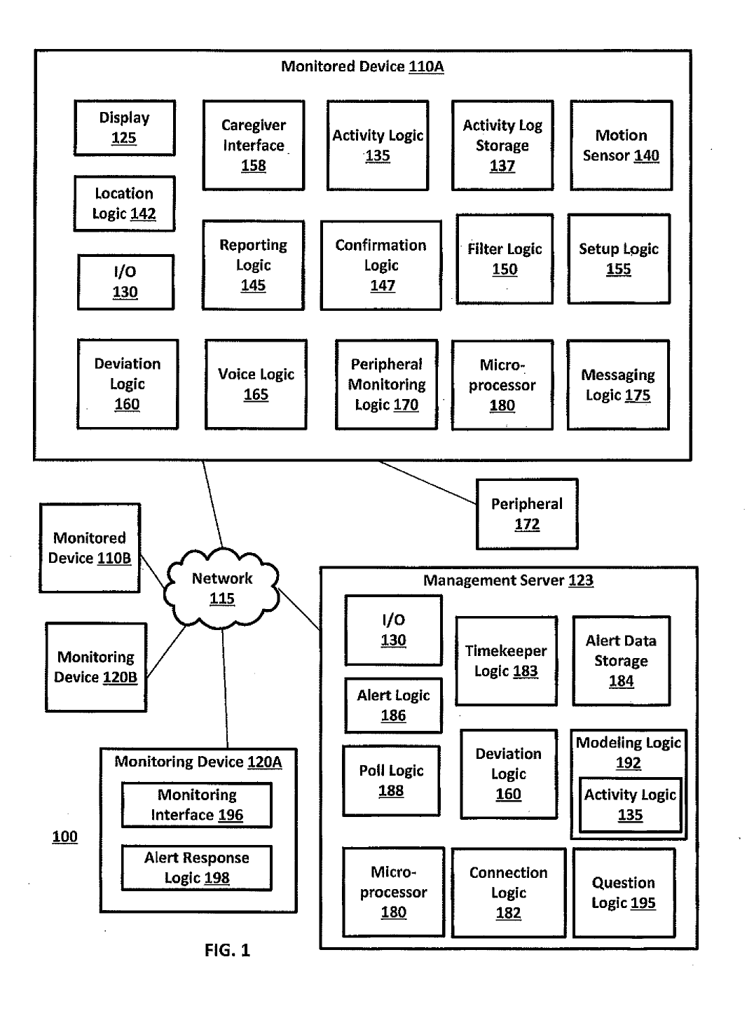

FIG. 1 illustrates a block diagram of a Monitoring System 100, according to various embodiments of the invention. The Monitoring System 100 is optionally configured to enable a (first) user of a Monitoring Device 120A to monitor activity of a (second) user of a Monitored Device 110A. In some embodiments the first and second users are peers, e.g., members of a family or social network. In some embodiments, the first user is a caregiver user, medical professional and/or employee of a care organization, and the second user is a cared for user such a senior, person with disabilities, child, patient, etc. A cared for user is a user receiving monitoring, assistance and/or medical care, and may be referred to herein as a “client” when the assistance or care is provided by a home care agency or other health care organization. A cared for user may also be referred to as a “monitored user.” A “caregiver user” is a user of Monitoring System 100 that aids and/or medical care to a monitored user. A caregiver user may be an individual homecare agent, an individual medical professional, or a care organization such as a nursing home, homecare agency, hospital, medical group, etc.

Monitoring System 100 is typically configured to make the activity monitoring simple and passive, while at the same time allowing the second user to protect their privacy and optionally to control how their activity is reported. As disclosed herein, the type of monitoring, activity monitoring and privacy controls may vary significantly between embodiments. Monitoring System 100 typically includes multiple Monitored Devices 110 and Monitoring Devices 120, individually referenced as 110A, 110B . . . and 120A, 120B, etc.

Activity monitoring typically occurs via a Network 115 and may be facilitated by a Management Server 123. Network 115 can include the Internet, a cellular network, a telephone network, a cable network, and/or any other network configured to communicate digital data. Management Server 123 is configured to manage activity monitoring between multiple users, e.g., users of multiple Monitored Devices 110 and/or multiple Monitoring Devices 120. Management Server 123 is optionally configured to maintain a social network between the multiple users. This social network establishes which users are followers (monitors) and which are monitored by followers. A user may be both followed and a follower. For example, a specific mobile device may be both an embodiment of Monitored Device 110A and a Monitoring Device 120A. Typical embodiments include many more Monitored Devices 110 and Monitoring Devices 120 than are illustrated in FIG. 1.

In an exemplary embodiment, Monitored Device 110A is configured to detect physical movement using an accelerometer and/or gyroscope. If physical movement is not detected for a predetermined amount of time, e.g., 15 hours. Then an alert indicating this fact is sent to Monitoring Device 120A. Management of the alert can be performed on Monitored Device 110A and/or Management Server 123. The conditions for, content of and criteria for alerts can vary widely and are optionally set by the users of Monitored Device 110A and/or Monitoring Device 120A.

Monitored Devices 110 can include, for example, a smartphone, a tablet computer, a personal computer, a wearable device (e.g., pet collar, necklace, shoe, watch, ring, bracelet, armband or medical sensor), a television set, smoke or CO₂ detector, a refrigerator, a microwave, a washer, a dryer, a coffee maker, body position sensor, television/internet interface device (e.g., Roku), a remote control, a telephone, a camera, a vehicle (e.g., car or truck), a stove, a light switch, a motion sensor, a door sensor, a security system, a light, and/or the like.

Monitored Device 110A optionally includes a Display 125 configured to present a user interface on Monitored Device 110A. Display 125 can include, for example, a touch screen of a smartphone, a security system control panel or a television screen. Display 125 is optionally configured for a user to enter commands or select controls. Users of Monitored Device 110A can include a monitored user (e.g., a patient senior a disabled person, a client of a care organization, and/or other cared for person). Users of Monitored Device 110A can also include a caregiver user (e.g., a caregiver, family member, employee of a care organization, medical professional, etc.)

Monitored Device 110A also includes an I/O (Input/Output) 130 configured to communicate data to and/or from Monitored Device 110A. I/O 130 can include, for example a cellular telephone transmitter, a WiFi interface, a Bluetooth interface, a Universal Serial Bus, an Ethernet connection, a coax interface, a fiber optic interface, and/or the like. I/O 130 is configured to communicate data using one or more communication channels. In some embodiments, I/O 130 is configured to communicate via TCP/IP or similar protocol.

Monitored Device 110A further includes Activity Logic 135. Activity Logic 135 is configured to detect use of Monitored Device 110A. In various embodiments, the detected use includes a wide variety of activities. For example, the detected use a can include one or any combination of: movement of Monitored Device 110A, use of Display 125, use of I/O 130, a location of Monitored Device 110A, horizontal deceleration of Monitored Device 110A, vertical deceleration of Monitored Device 110A, charging/power level of Monitored Devices 110A, WiFi connection made by Monitored Device 110A, use of a camera included in Monitored Device 110A, use of peripheral device having a direct wired or wireless connection to Monitored Device 110A, execution of a software or hardware application on Monitored Device 110A, detection of sound by Monitored Device 110A (e.g., monitoring of an audio environment), use of Monitored Device 110A to make or receive calls, video sent by Monitored Device 110A, detection of movement in an environment near Monitored Device 110A, detection of signals by Monitored Device 110A, measurements made by Monitored Device 110A, control of objects by Monitored Device 110A, use of a communication channel (e.g., cellular telephone connection, an internet connection, a wireless connection, etc.) to communicate from Monitored Device 110A, and/or the like. Movement of Monitored Device 110A is optionally detected using a gyroscope, an accelerometer, a galvanometric sensor, barometer, a local positioning system, and/or a global positioning system. The detected use of Monitored Device 110 optionally includes the presence of Monitored Device 110 at a geographic location. Importantly, the detected use optionally includes a lack of use in specific time period.

As used herein, the term “area of regard” is meant to indicate the area in which a sensor can detect movement or other activity. For example, a camera or infrared motion detector may have a “field of view” in which movement can be detected. This field of view would be considered the “area of regard” for these sensors. A pressure sensor under a carpet has an area of regard that includes the part of the carpet on which it may detect a step. Window and door sensors have an area of regard that typically includes the opening and closing of the window or door. Internet of Things (IoT) devices may include sensors whose area of regard includes a use of that specific device, e.g., opening a refrigerator door or adjusting a thermostat.

Monitored Device 110 optionally further includes a Motion Sensor 140. Motion Sensor 140 is configured to detect movement of Monitored Device 110 and/or movement within an environment around Monitored Device 110. For example, Motion Sensor 140 can include a gyroscope and accelerometers such as those commonly found in smartphones or table computers. In another example, Motion Sensor 140 includes a camera and logic configured to detect movement in images recorded by the camera, an ultrasonic sensor, microphone, a pressure sensor, an electric eye, an infrared sensor, a light sensor, a vibration sensor, a magnetic sensor, a mechanical sensor, a radar device, and/or the like. Motion Sensor 140 may be configured to detect motion of a person and/or an object. For example, Motion Sensor 140 may be configured to detect movement of a person within an area of regard, opening or closing of a door, etc. Motion detected by Motion Sensor 140 is reported to Activity Logic 135 and may be considered to reflect a use of Monitored Device 110.

In some embodiments, one, two or more of Motion Sensors 140 are disposed external to Monitored Device 110A. Each of these Motion Sensors 140 typically has a different area of regard in which they can detect motion. For example, one of Motion Sensors 140 may be placed in a bathroom and another of Motion Sensors 140 may be placed in a hallway. In combination, they can detect movement (and lack thereof) in both these spaces. Motion Sensors 140 are optionally disposed in a peripheral device, such as a wearable device worn by a monitored user. As used herein, “use of Monitored Device 110A” is intended to include use of or signals generated by external instances of Motion Sensor 140 and use of or signals generated by instances of Peripherals 172.

Monitored Device 110 optionally further includes a Location Logic 142. Location Logic 142 is configured to determine and report a location of Monitored Device 110A and/or a location of a peripheral device having a wired or wireless connection to Monitored Device 110A. The determination can include interpretation of a signal from a global positioning system (GPS), for mapped Wi-Fi signals, from a local positioning system, from inertial measurements, from beacons (e.g., Bluetooth beacons), and/or the like. Location Logic 142 can include an antenna and/or timing circuit configured to receive wireless signals used to determine location.

In some more specific examples, Activity Logic 135 is configured to detect the use of Monitored Device 110A including the following:

1) Presence or absence of Monitored Device 110A at a specific location, optionally as determined by Location Logic 142. The specific location can be a hospital, police station, employment location, a school, a city, a state, a care facility, and/or the like. For example, Monitored Device 110A may be detected to be at a hospital or not to be at a school. The location may be a critical location such as a hospital or police station.

2) Use of Peripheral Device 172 coupled to Monitored Device 110A. Peripheral Device 172 may be coupled using a wireless or direct wired connection. For example, Peripheral Device 172 may be coupled to Monitored Device 110A using Bluetooth or WiFi. The use of Peripheral Device 172 can include any of the uses of Monitored Device 110A discussed herein. In some examples, the use of Peripheral Device 172 includes the detection of signals or collection of data using sensors of Peripheral Device 172. For example, use of Peripheral Device 172 can include detection of movement using a camera, radar or ultrasonic movement sensor. Use of Peripheral Device 172 can include detection of a door opening or closing, operation of a vehicle or reading of an RFID tag. Use of Peripheral Device 172 can include detection of a person's actions, movement (e.g., steps) or vitals. For example, the detected use may include opening of a medication container, movement of a wearable device, that a person has visited a bathroom, water flow, a blood glucose measurement, how much a person walks, a gait, a blood pressure measurement, an EKG, use of a medical device, and/or the like.

3) Measurement of a physical activity of a user. For example, the use may include a person's steps taken, respirations or heart rate as measured by a wearable device. The use may include changing channels or inputs sources on a television or video monitor, optionally via a remote control. The use may include a signal detected from a pressure sensor in a bed or on a floor.

4) Movement of Monitored Device 110A, including for example, being picked up, turned over, and/or the like. The detected use can include quantitative measurement of acceleration in specific directions.

5) Operation of Monitored Device 110A, including, turning on/off, execution of software and/or hardware applications on Monitored Device 110A. These can include use of a camera, microphone, speaker, and/or display. In some embodiments, the detected use of Monitored Device 110A includes predicting and reporting a loss of power to Monitored Device 110A. For example, Monitored Device 110A may be configured to provide a location prior to losing power.

The use of Monitored Device 110A, as detected by Activity Logic 135, is optionally stored in an Activity Log Storage 137. Activity Log Storage 137 includes a non-transient digital storage such as random-access memory, static memory, optical memory, a hard drive, flash memory, and/or the like. Activity Log Storage 137 optionally further includes a data structure configured to store data records of use activity. Activity Log Storage 137 is optionally additionally or alternatively disposed on Management Server 123. In an illustrative embodiment, Activity Log Storage 137 is configured to store a record of a time a last movement was detected using Activity Logic 135 and Motion Sensor 140 (or Location Logic 142). This movement can be of Monitored Device 110A or of an object with an environment of Monitored Device 110A. This movement can be small, e.g., Monitored Device 110A was merely picked up or turned over. This movement can be larger, e.g., as may be detected by Location Logic 142.

Monitored Device 110A further includes Reporting Logic 145. Reporting Logic 145 is configured to report detected use of Monitored Device 110A to a remote destination, e.g., to Management Server 123 and/or directly to Monitoring Device 120A. The remote destination can be a cloud-based data storage facility. The remote destination is optionally managed by a third party. Reporting Logic 145 may report details of all detected use, an abstracted representation of detected use, a summary of detected use (e.g. a “digest”), and/or a filtered version of detected use. The information reported is optionally selected by a user of Monitored Device 110A using Setup Logic 160 discussed elsewhere herein. Further, in some embodiments, Reporting Logic 145 only reports detected use of Monitored Device 110A if certain criteria are met, and/or the content of information reported may be dependent on meeting some criteria. The information reported may be extracted from other data. For example, several footsteps might be extracted from accelerometer data, walking distance might be extracted from footsteps, a car crash extracted from horizontal accelerometer data, a summary over time might be extracted from discrete events, etc. These extractions may be subject to privacy settings selected by a user of Monitored Device 110A.

In some embodiments, sensor data is partially processed on Monitored Device 110A and then communicated to Management Server 123 for further processing. For example, acceleration data indicative of a traffic accident or a dropped phone may first be analyzed first on Monitored Device 110A by Activity Logic 135, the results of this analysis may be then sent to Management Server 123 by Reporting Logic 145 for further analysis, if the acceleration data is likely to represent a reportable event. At Management Server 123 more thorough analysis of the data may confirm a traffic accident or dropped phone. In a more specific example, Activity Logic 125 may be configured to detect sharp acceleration events (as detected by an accelerometer), only when these events are above a threshold are, they reported to Management Server 123. At Management Server 123 the acceleration events are further analyzed, perhaps using a machine-based system to confirm whether they are indicative of a reportable event. The machine-based system optionally includes, for example, a machine learning system trained to identify acceleration patterns as would be expected in a traffic accident. Separate machine-based systems are optionally used to identify specific activities based on sensor data and to determine a health state or other condition based on the identified specific activities. These machine-based systems may be located on different devices, e.g., Monitored Device 110A and Management Server 123. All or part of Activity Logic 135 can be disposed on Management Server 123. As used herein the term “health state” is used to refer to a person's mental and/or physical state of health. An undesirable health state is one that is likely to have negative health consequences.

Reporting Logic 145 may be configured to report that Monitored Device 110A has not been moved(or no movement detected by Motion Sensor 140) for more than 8 hours during the daytime (or 13 hours at nighttime), to report that Monitored Device 110A experienced a rapid horizontal deceleration such as would be detected during a vehicle accident, to report that Monitored Device 110A is unexpectedly at a hospital, to report that. Monitored Device 110A did not go to a grocery store for over a week, to report that Monitored Device 110A did not go to church on Sunday, to report that Monitored Device 110A left a geographic region, and/or the like. Reporting Logic 145 may be configured to report deviations from expected use of Monitored Device 110A as determined by Deviation Logic 160, which is discussed further elsewhere herein.

In some embodiments, Reporting Logic 145 is configured to respond to a polling signal from Management Server 123. The response can be a simple “I'm alive” response or a response that include more data regarding the state of Monitored Device 110A. For example, Reporting Logic 145 may be configured to response to a poll from Management Server 123 by providing a report of the use of Monitored Device 110A to Management Server 123.

Alternatively, Reporting Logic 145 may be configured to push reports of activity to Management Server 123. The pushed reports may be sent on a regular basis (e.g., at a fixed frequency or period), sent in response to specific activity detected using Activity Logic 135, and/or sent in response to a specific command from a user of Monitored Device 110A. A report is considered an “alert,” e.g., a communication configured to alert a user of Monitoring Device 120A of a use (or lack thereof) of Monitored Device 110A, if the report is triggered in response to a specific detected use. Such an alert may be triggered and generated based on any of the uses detected by Activity Logic 135 discussed herein, e.g., Monitored Device 110A has not moved in 14 hours or just experienced a rapid deceleration. The alert may include an identity of Monitored Device 110A, a user's identity, and/or the use of Monitored Device 110A on which the alert was based. Alerts are triggered as a result of specific events (or lack thereof) and may be sent automatically without requiring further action by the user being monitored or a follower of the user. In contrast, digests are typically sent at regular time periods chosen by a user and are sent without being triggered by a specific event (or lack thereof). Embodiments of the invention include either or both alerts and digests.

In some embodiments, Reporting Logic 145 is configured to provide a user of Monitored Device 110A with an opportunity to prevent an alert from being communicated to Monitoring Device 120A. For example, a Confirmation Logic 147 is optionally configured to confirm that an alert should be sent to Monitoring Devices 120. Confirmation Logic 147 is configured to provide notice to a user of Monitored Device 110A that an alert will soon be sent. For example, Confirmation Logic 147 may cause a message to appear on Display 125, may cause Monitored Device 110A to make a sound, may send a text message to Monitored Device 110A, and/or may make a telephone call to Monitored Device 110A. All or part of Confirmation Logic 147 is optionally disposed on Management Server 123.

For example, in some embodiments, Confirmation Logic 147 is configured to present a notice on Display 125. The notice including a message that an alert is about to be sent, contents and/or reasons for the alert (e.g., a deviation from an expected use of Monitored Device 110A), and/or an opportunity to confirm or cancel the alert. The opportunity to confirm or cancel can include a command input (e.g., virtual button). By way of example, a message may be displayed saying that an alert will be sent in 2 minutes because Monitored Device 110A has just detected an abnormal sugar level via an insulin pump. The display includes a button labeled “Cancel,” activation of which would cancel the pending alert. The amount of time given to cancel the alert is optionally dependent on the cause of the alert. For example, a short period may be given if Monitored Device 110A has experienced deceleration as would be expected in a car accident while a longer period may be given if Monitored Device 110A has not been moved at all for 12 hours. Confirmation Logic 147 is optionally configured for a user to provide information that would be included in an alert. For example, if the alert is generated because Monitored Device 110A is at a hospital, the user may add a comment “just picking up medication” or “David hit his thumb with a hammer.”

Monitored Device 110A optionally further includes Filter Logic 150. Filter Logic 150 is configured to summarize use detected by Activity Logic 135. One purpose of Filter Logic 150 is to enhance the privacy of Monitoring System 100, with respect to the user of Monitored Device 110A by applying a privacy filter. The action of Filter Logic 150 can prevent the user from feeling that their every action is being monitored or watched. Specifically, by summarizing detected movement of Monitored Device 110A the only information that need be reported is when the movement triggers an alert rule, which can be set by the user of Monitored Device 110A. For example, the user may select (or agree to a default) that a lack of movement for 15 hours overnight is grounds for an alert. In this case, Filter Logic 150 is configured to eliminate detailed movement data and just report that Monitored Device 110A has not been moved for 15 hours. Filter Logic 150 restricts the information that is included in reports/alerts that are sent by Reporting Logic 145. In typical embodiments, the filtering performed by Filter Logic 150 is configurable by the user of Monitored Device 110A. For example, they may select what actions would cause an alert to be sent, what is included in an alert, and/or a subset of Monitoring Devices 120 to which alerts are sent. For example, the user may have alerts that relate to medical conditions, being at a police station etc. sent to close family members and alerts that involve lack of movement of Monitored Device 110A or activation of a home alarm system, to be sent to friends. A user may specify that specific types of uses not be reported by specifying a privacy filter.

In some embodiments, Filter Logic 150 is further configured to generate digests of a user's activity. Filter Logic 150 is optionally disposed on Management Server 123 in addition to or as an alternative to Monitored Device 110A. In this case, part of the filtering is performed on Management Server 123. In some embodiments, Filter Logic 150 is configured to convert an image to data such as privacy is protected.

Monitored Device 110A optionally further includes a Setup Logic 155. Setup Logic 155 is configured for a user (caregiver user or monitored user) of Monitored Device 110A to customize operation of Activity Logic 135, Reporting Logic 145, Filter Logic 150, and/or other logic within Monitored Device 110A (as discussed elsewhere herein). For example, Setup Logic 155 may be used to select which use of Monitored Device 110A is included in reports sent by Reporting Logic 145. In some embodiments only use selected by the user of Monitored Devices 110A is reported to third parties. Setup Logic 155 is optionally configured for a user of monitored Device 110A or Monitoring Device 120A to select which detected use of Monitored Device 110A is reported to Management Server 123 or Monitoring Device 120A. Setup Logic 155 is optionally further configured for the user of Monitored Device 110A to select which of Monitoring Devices 120 are to receive alerts in response to Reporting Logic 145, e.g., to select who is permitted to be a follower of the user. Setup Logic 155 is optionally configured to customize operation of Monitoring System 100 on a follower by follower basis, allowing different followers to receive different information.

Examples of the customization for which Setup Logic 155 may be configured include, but are not limited to:

Setting periods for which non-movement and/or lack of other use of Monitored Device 110A would result in an alert. These periods may be dependent on time of day and/or day of the week.

Setting locations at which the presence of Monitored Device 110A would cause an alert. For example, a hospital, police station, airport, travel out of county or out of state, doctor's office, and/or the like.

Setting locations at which absence of Monitored Device 110A would cause an alert. For example, not being at a church on Sunday morning, not being in a class room at an expected time, not being at work during worktime, not being along a delivery route when expected, not being at an appointment, and/or the like. Appointment times may be determined by calendar integration.

Setting connections with, and alert rules for, peripheral devices such as Peripheral 172. The “alert rules” being rules that result in an alert when broken. E.g., send an alert if blood glucose is detected to be above 300.

Setting privacy criteria that specify criteria for protecting the privacy of users. The criteria limiting, for example, what use of Monitored Device 110A can be shared with specific Monitoring Devices 120.

Acceptance of followers.

Setting parameters that define expected use of Monitored Device 110A.

Selecting apps (executing in Monitored Device 110A) whose use is monitored by Activity Logic 135.

Setting the content of alerts under various conditions.

Entry of a care plan and/or post-operative plan associated with a cared for user.

All or part of Setup Logic 155 may be disposed on Management Server 123. In this case, the user of Setup Logic 155 may access Setup Logic 155 via a browser or other application on Monitored Device 110A or on a separate computing device.

Monitored Device 110A optionally further includes Deviation Logic 160. Deviation Logic 160 is configured for determining if use of Monitored Device 110A, as detected by Activity Logic 135, deviates from an expected use of Monitored Device 110A. The detection of the deviation typically results in generation of a report by Reporting Logic 145. The expected use may be defined by alert rules discussed elsewhere herein. The deviation in use may include a temporal and/or a spatial deviation. For example, a use may be time based (e.g., no activity for a day) or location based (presence in an unexpected space)

The deviations detected by Deviation Logic 160 optionally include more than merely violation of alert rules. The deviations can include deviations from patterns of activity. For example, if Monitored Device 110A is normally taken to a grocery store at least once a week, a failure to do so for two weeks may be grounds for a report to Management Server 123 or Monitoring Device 120A. If the user of Monitored Device 110A normally walks for an hour each day (as detected by Motion Sensor 140 or Location Logic 142, a reduction to 10 minutes of walking per day may be grounds for a report and alert.

The “expected use,” to which detected use is compared, may be defined manually by a user of Monitored Device 110A and/or Monitoring Device 120A. The expected use may be defined by predefined (e.g., default) alert rules. Further, the expected use may be defined by looking for and detecting past use patterns. For example, Deviation Logic 160 may be configured to detect a pattern (e.g., frequency, times, etc.) at which the user visits a pharmacy, visits church, goes to work or school, exercises, makes bathroom visits, sleeps, and/or other class of activity. The detected pattern can be based on the activity of a specific individual being followed, on a group of people having similar characteristics (e.g., women between 60 and 65 years old), on the general population, and/or any combination thereof. For example, an expected use for everyone would be to use the restroom at least once a day, an expected use of women between 60 and 65 may be walking at least 3000 steps a day, and an expected use for a specific person would be to go to her bridge club on Tuesday evenings. The expected use for an individual may change over time. As used herein “expected use” of Monitoring Device 110A is meant to include the expected activity of a person monitored using Monitoring Device 110A. For example, an expected use can include receipt of data from an IR sensor (Peripheral 172) that reports movement of an individual. An expected use may be embodied in a trained machine learning system whose output represents one or more measures between an actual use received as input and the expected use. The amount of deviation from expected use is optionally represented by this output.

In some embodiments, Deviation Logic 160 includes a machine-based system. As used herein a “machine-based system” is used to specifically mean: an artificial neural network, an artificial intelligence, a Bayesian statistical system, a deep learning system, a machine learning system, a rule-based system, an expert system, and/or the like. The machine-based system is optionally configured to learn (e.g., is trained) from received data. For example, an output of a neural network may be improved using data generated by Monitored Device 110A. The output may include detection that a later use of Monitored Device 110A deviates from patterns detected in the past use. The past use is optionally based on logs of use stored in Activity Log Storage 137. In some embodiments, the machine-based system is trained based on multiple Monitored Devices 110A used by the same user or by multiple different users, respectively. As used herein a “machine learning system” is meant to indicate a “machine-based system” configured to learn or be trained using received data. As an example, a hard-coded rule-based system or purely statistical system would be a machine-based system but not a machine learning system. A neural network whose output improves by the addition of quality training data would be considered a machine learning system.

When Deviation Logic 160 detects a use that deviates from the expected use, Reporting Logic 145 is optionally configured to report this detected deviation. As a result, a corresponding alert may be sent to one or more of Monitoring Devices 120 and/or Management Server 123.

Monitored Device 110A optionally further includes Caregiver Interface 158. Caregiver Interface 158 includes computing instructions and/or other logic configured for a caregiver to communicate with Monitored Device 110A and Management Server 123. Caregiver Interface 158 can include input devices, such as a camera, microphone, keyboard or touchscreen, and is typically configured to display a graphical user interface on Display 125. Caregiver Interface 158 optionally includes login logic configured for a caregiver to login to Monitored Device 110A. The login can include, for example, entry of a username/password combination, scanning of a barcode, facial recognition, detection of a wireless signal (e.g., RFID tag, Bluetooth or WiFi device in possession of the caregiver). Logging in and out of Monitored Device 110A is optionally used for tracking a caregiver's clocking in and out for a shift. For example, the time a caregiver logs in can be used as a shift start for pay and work hour reporting, while a time the caregiver logs out can be treated as the end of a shift.

Caregiver Interface 158 can be configured to perform a wide variety of functions. For example, Caregiver Interface 158 can be configured to present a care plan for a user (e.g., monitored senior) to the caregiver. The caregiver can check-off or otherwise acknowledge which aspects of the care plan have been completed at a visit. Caregiver Interface 158 can also be used by caregivers to enter notes and read notes provided by other caregivers caring for the same user. These notes may be added to a client's records and/or communicated to a care agency.

In some embodiments, Caregiver Interface 158 is configured to provide questions to caregivers regarding a monitored user. As discussed elsewhere herein, these questions are optionally selected in response to sensor data and/or detected activities of the user. Caregivers can report/provide answers to the questions via Caregiver Interface 158 via, typing, speaking into a microphone, or checking boxes. The answers can be provided by the user in response to the caregiver asking the user a question or may be based on observations of the user by the caregiver. For example, a question of “How did you sleep last night?” can generate an answer from the user that is entered by the caregiver in Caregiver Interface 158, or a question “Does Ms. Clark look rested today?” can generate an answer about the caregiver's direct observations/opinion. The provided answers are optionally used to select follow-up questions. Caregiver Interface 158 is optionally configured such that Monitored Device 110A serves to: provide a timekeeping interface for caregivers, provide a digital checklist for tasks to be performed by a caregiver during a care visit, provide note sharing functions for care notes to be communicated between caregivers and/or their agency, provide questions to which a caregiver can respond with answers about a cared for user.

In Caregiver Interface 158, checkboxes can be used to provide answers to Yes/No questions or questions such as “on a scale of 1 to 5 . . . ” In some cases, a caregiver may be asked to add further details to an answer depending on which box is checked. In some embodiments, Caregiver Interface 158 is configured to record and send an image as an answer to a question. For example, a question may include “please record an image of Ms. Smith's sutures,” and a camera on Monitored Device 110A or Peripheral 172 can be used to record the image as an answer. Such a process may be used to generate a series of images for examination by medical personnel or examination using Activity Logic 135 and/or Activity Analysis Logic 730. The inclusion of images within answers may be used to monitor, for example, wound healing, changes in a skin blemish (possibly skin cancer), edema, muscle tone, facial symmetry, injuries, bruises, suspected abuse, insect bites, eye injuries, infections, and/or the like.

Monitored Device 110A optionally further includes Voice Logic 165. Voice Logic 165 is configured to activate a voice channel between Monitored Device 110A and Monitoring Device 120A. For example, Voice Logic 165 may be configured to automatically initiate a call using the mobile computing features of Monitored Device 110A and/or Monitoring Device 120. This call may be in response to a command received from a remote source. For example, in response to an alert received at Monitoring Device 120A, a command may be sent to Voice Logic 165 on Monitored Device 110A. In response to receiving this command, Voice Logic 165 may be configured to place a telephone call (POTS or IP) from Monitored Device 110A and optionally turn on a “speaker” mode.

The command sent by Monitoring Device 120A optionally includes authentication data or a security certificate configured to prevent Voice Logic 165 from being used to initiate unauthorized calls. This security feature may be uniquely associated with the corresponding alert.

Monitored Device 110A optionally further includes Peripheral Monitoring Logic 170. Peripheral Monitoring Logic is configured to monitor and received data from one or more Peripheral 172. Peripheral Device 172 can include, for example, a vehicle, a wearable device, an earring, a defibrillator, a necklace, a shoe, a medical device, a medicine delivery device, a medication container, an insulin pump, a pacemaker, a wearable activity sensor (e.g., Fitbit® or AppleWatch®), a blood/oxygen level detector, a pulse measurement device, a blood pressure measurement device, a prosthetic, a mouse, a keyboard, a camera, an (external) Motion Sensor 140, a vehicle control system, a remote control, a set-top box, an electronic door lock, a door or window sensor, a pressure sensor, a physical security system, headphones (earbuds), a radar system, and/or the like. For example, in some embodiments Monitored Device 110A includes a computing device, such as a tablet computer, connected to the internet, and configured to receive sensor data from multiple Peripheral 172 via wired or short-range wireless connections.

In some embodiments, Peripheral Monitoring Logic 170 is configured to received data from one or more Peripheral 172 and determine if the data represents an abnormal condition, e.g., an abnormal use of Monitored Device 110A. As noted elsewhere herein, use of Peripheral 172 and/or data received therefrom, can be considered an example of use of Monitored Device 110A. Peripheral 172 and Monitored Device 110A may be connected via a wire, optically, and/or via a wireless connection. For example, in one embodiment Monitored Device 110A is an iPhone® and Peripheral 172 is an Apple Watch® connected by wireless Bluetooth®.

In some embodiments, Monitored Device 110A further includes Messaging Logic 175. Messaging Logic 175 is configured to communicate at least text messages (SMS or MMS) between the Monitored Devices 110 and Monitoring Devices 120. For example, a user of Monitoring Device 120A may send texts or images to Monitored Device 110A. In some embodiments, Messaging Logic 175 is configured to display received images using Display 125. These received images (or texts) may appear sequentially or randomly on Display 125 as occurs on an electronic picture frame. The texts or images are optionally sent in response to a daily digest received by Monitoring Device 120A.

Management Server 123 may be configured to both manage activity monitoring between multiple Monitored Devices 110 and multiple Monitoring Devices 120, and manage a social network between the users of these devices. The social network optionally includes more than one type of connection. For example, in some embodiments, the social network includes a basic connection and an enhanced connection. The basic connection is like those found in social networking applications such as Facebook®, LinkedIn® and Instagram®. These connections allow manual sharing of content, messaging and automatic acknowledgement that a message was received. The enhanced connections provide the functionality of the basic connections and in addition provide the automated monitoring functions described herein. For example, the enhanced connection may provide automatic alerts of deviations from the expected use of Monitored Device 110A.

Management Server 123 includes an embodiment of I/O 130. This embodiment is configured to communicate via Network 115 to multiple instances of Monitored Devices 110 and Monitoring Devices 120.

Management Server 123 further includes Connection Logic 182. Connection Logic 182 is configured to establish and maintain social connections between users of Monitored Devices 110 and Monitoring Devices 120, and optionally other types of network access devices. The connections can be uni-directional, e.g., from a follower to a person being followed, or bi-directional. A person who is a followed can also be a follower. Embodiments of Connection Logic 182 are optionally included in Monitored Devices 110.

Connection Logic 182 is typically configured to establish connections between people. For example, a first person may request to follow a second person, following approval by the second person, a connection is established. The second person can set alert rules, privacy criteria and the like using Setup Logic 155. The rules and criteria may be applied to all followers of the second person or to a specific follower. Thus, alert rules and privacy criteria can be specified on an individual basis if desired. Part of Setup Logic 155 may be disposed within Connection Logic 182 for this purpose.

Connection Logic 182 may also be configured to establish which Monitored Devices 110 are associated with which users. For example, a user may have a smartphone, tablet computer, home security system, and personal glucose sensor. The user may use Connection Logic 182 to identify which Monitored Devices 110 are to be monitored on their behalf. Further, Connection Logic 182 may be configured to identify which Monitoring Devices 120 alerts should be sent to. For example, a follower may designate that alerts should be sent via text message to a cell phone and via e-mail to an e-mail account.

Connection Logic 182 is optionally configured to provide basic social networking functions such as those found on FaceBook® or Instagrame. These functions include sharing of content, messaging between connections, and the like.

In some embodiments, Connection Logic 182 is configured to manage a social network having at least two different types of connections between members. These connections include a basic connection having the basic social networking functions of the prior art, and an enhanced connection that includes the monitoring of user activity and generation of alerts when use of Monitored Device 110A meets the criteria for sending an alert to Monitoring Device 120A. A managed social network optionally includes at least a basic connection between all connected members, and an enhanced connection between a subset of the connected members.

In some embodiments, Connection Logic 182 is configured for a member of Social Network 200 to upgrade a connection to another member from a Basic Connection 210 to an Enhanced Connection 220. Such an upgrade may be suggested by a manager of Social Network 200 and may involve a fee or other consideration. For example, the manager of Social Network 200 may suggest an upgrade between members of Social Network 200 that are identified as family members. The manager may be Facebook, Inc. or LinkedIn, Inc. etc.

In some embodiments, Connection Logic 182 is configured for a user to register multiple Monitored Devices 110 to a single account. For example, the user Artemis 230C may have a smartphone, a tablet computer, and additional personal electronic devices, each of which is a Monitored Device 110. The use of all the registered devices registered to Artemis 230C's account can be used to monitor her activity in parallel.

The use of Connection Logic 182 to create an Enhanced Connection 220 optionally includes using Setup Logic 155 to establish alert rules and privacy criteria for the Enhanced Connection 220. All or part of Setup Logic 155 may be disposed on Management Server 123. For example, Artemis 230C may request to follow Sophie 230A in an Enhanced Connection 220 (an upgrade of their existing Basic Connection 210). Connection Logic 182 is configured to send Sophie 230A an option of accepting the request from Artemis 230C. If Sophie 230A accepts the request, Connection Logic 182 typically directs Sophie 230A so that she can choose how information regarding her activities is provided to Artemis 230C in activity alerts. Optionally, a selection of default privacy levels is provided, each of which can be further customized.

Management Server 123 optionally further includes Timekeeper Logic 183, Timekeeper Logic 183 is configured to receive shift check-in and check-out times from caregivers, and to store these times for payroll purposes. These times may be entered by the caregivers via Caregiver Interface 158 as discussed elsewhere herein. Timekeeper Logic 183 may also be configured to receive time of day data regarding the completion of caregiver tasks for a cared for client. The operation of Activity Logic 135 is optionally dependent on whether a caregiver is in a user's residence. For example, when a caregiver checks in for a shift, sensor data generated by Motion Sensor 140 and/or Peripherals 172 may be analyzed considering that detected motion may be that of the caregiver rather than the user.

Management Server 123 typically further includes an Alert Data Storage 184 configured to store alert rules, privacy criteria, and/or any other data indicating under which conditions an alert should be sent to one or more members of Monitoring Devices 120. This data stored in Alert Data Storage 184 may include default rules/criteria and/or rules/criteria specified using Setup Logic 155. Alert Data Storage 184 optionally further includes account information of users of Monitoring Devices 120. This account information can include enhanced connections (discussed elsewhere herein), names, telephone numbers, e-mail addresses, IP addresses, MAC addresses, and/or the like. Such account information can be used to direct alerts to the proper locations. Alert Data Storage 184 optionally further includes a log of alerts sent and/or records of use received from Monitored Devices 110. Alert Data Storage 184 optionally includes data structures specifically configured for storing the data discussed herein.

Management Server 123 typically further includes Alert Logic 186 configured to provide an alert to one or more of Monitoring Devices 120. The same alert is optionally sent to multiple Monitoring Devices 120. The alert is sent in response to data characterizing use of the first of one or more Monitored Devices 110. For example, an alert may be sent in response to use data received from Reporting Logic 145. Alert Logic 186 may be configured to compare received use data with alert rules stored in Alert Data Storage 184, and if the actual use represented by the received use data violates a rule that an alert may be sent. The contents and the recipients of the alert are governed by privacy criteria. Specifically, if the actual use of Monitored Device 110A is found to be outside of the expected use, e.g., is an abnormal use, then an alert is generated by Alert Logic 186. The abnormal use can, of course, be a lack of use. The use data is received from Monitored Device 110A because the user has an enhanced connection with at least one follower, e.g., with a user of Monitoring Device 120A.

In some embodiments, Alert Logic 186 is configured to associate multiple Monitored Devices 110 with a single user and to apply alert rules to the Monitored Devices 110 as a group. For example, if an alert rule includes that a device be moved or touched at least once a day, that rule could be applied such that at least one of a group of devices is moved or touched then the use is within the “expected use.” As applied, this means that if a user has a smartphone, a tablet computer, a monitored vehicle and a front door motion sensor, the activation/movement/use of any of these devices indicates that the user is doing things (e.g., not sick or fallen) and an alert need not be sent. Only if none of these devices is used is an alert sent. Such relationships between Monitored Devices 110 are optionally established using Setup Logic 155 on Management Server 123 or Monitored Device 110A. In some embodiments, Alert Logic 186 is configured to compare the alert data to the data characterizing the use of the first of the remote clients and to provide the alert in response to this comparison.

Alert Logic 186 is optionally configured to use Deviation Logic 160 disposed on Monitored Device 110A and/or Management Server 123. As discussed elsewhere herein, Deviation Logic 160 is configured to determine if an actual use of one or more of Monitored Devices 110 deviates from an expected use.

Management Server 123 optionally further includes Modeling Logic 192. Modeling Logic 192 is configured to model the use of one or more Monitored Devices 110. Modeling Logic 192 optionally includes embodiments of Activity Logic 135. The model is configured for distinguishing between expected use and a use that is not expected, e.g., deviates from the expected use. Modeling Logic 192 is optionally configured to train a machine-based system to distinguish between the expected use and the use that is not expected. Modeling Logic 192 is optionally configured to train the artificial intelligence system based on use of multiple monitored devices used by different users. Modeling Logic 192 is optionally configured to train the artificial intelligence system based on a specific user and a resulting trained artificial intelligence system is customized to the specific user. In a specific example, Modeling Logic 192 may start with a machine-based system trained based on the activities of a multiple users, and then further train this machine-based system based on the historic activities of a user. The multiple users may include a group to which the user is a member. For example, if the user may be a member of a group consisting of men 55-60 years old that are former professional athletes, or the particular user may be a member of a group consisting of female college students of a specific race. A wide variety of criteria can be used to define such groups. A model produced by Modeling Logic 192 can be used by Deviation Logic 160 to determine if actual usage of Monitored Device 110 is abnormal.

Management Server 123 optionally further includes Question Logic 195. Question Logic 195 is configured to select questions to be sent to caregivers via Caregiver Interface 158, and to receive answers to these questions. The received answers may be used to annotate sensor data and/or detected activities. The received answers may also be used to further train elements of Activity Logic 135 to determine activities from sensor data and/or to further train elements of Activity Logic 135 to determine a health state based on determined activities. The received answers may also be used to train a machine learning system within Question Logic 195 to select more valuable questions. The value of a question being based on how likely an answer to the question will be useful in reaching a goal, e.g., is predicted to improve a statistical accuracy of the machine learning system by the greatest degree. The value may be weighted by both the expected improvement and by the perceived value of correctly determining health states relative to others. For example, a more valuable question can be one that is more likely to determine a health state, to exclude possibility of a particularly undesirable health state, likely to be most useful for training one or more parts of Activity Logic 13S or Question Logic 195, and/or some other goal.

In various embodiments, Question Logic 195 is configured to select questions from a predefined set of questions. The predefined set may be generated in response to human input and/or a review of instances in which Monitoring System 100 failed to detect a health state. Question Logic 195 can be configured to use any combination of the criteria disclosed herein to select questions for presentation via Caregiver Interface 158.

In various embodiments, Question Logic 195 is configured to select questions based on data generated by Motion Sensors 140, Sensor 715A, and/or Peripheral 172. For example, data indicating that a user has not been opening a refrigerator can result in the selection of questions related to how a user has been eating and/or if the user is having trouble getting to the kitchen. In another example, sensor data indicating that a television volume selection is slowly getting higher can result in the selection of questions regarding hearing. In another example, if sensor data indicates that a user is spending more time in the bathroom than expected, then questions regarding urination or bowel movements may be selected. In another example, if sensors detect a shuffling gait, then questions regarding balance and falls may be detected. In another example, if sensors detect a general decline in activity, then questions regarding general feelings of health or psychological state may be selected.

In some embodiments, Question Logic 195 is configured to select questions based on a determined activity of a user, the activity typically being determined based on sensor data. For example, sensor data may be used to determine movement in a hallway or bedroom, heart rate, opening of a window, movement in a kitchen, television remote use, mobile device use, computer use, walking motion (e.g., number of steps, step speed, step height, step asymmetry, etc.) laying down, sitting, standing, sudden movements, any other examples discussed herein, and/or the like, as well as time spent in these activities. This sensor data may then be used to determine specific activities, or to determine directly a health state, using Activity Logic 135. For example, the above sensor data may be used to determine activities including walking down the hallway, yawning, going to bed, exercise, standing up, eating, television watching, reading a Facebook page, a shuffling walk or lack of balance, a nap, a fall, any other examples discussed herein, and/or the like.

The determined activities (or health state) can be used by Question Logic 195 to select specific questions. For example, activities of getting up several times during the night can lead to the selection of questions such as: “Did you sleep well?”, “Did you have to go to the bathroom a lot last night?”, “Are you tired?”, “Are you feeling stressed this morning?”, “Are you sleeping better?”, and/or the like. An activity of an uneven or asymmetric gait can result in the selection of questions such as: “Did you have some extra drinks last night?” “Are you feeling tired or having dizzy spells?” and/or “Have you felt like you might fall in the last couple of days?”

In some embodiments, Question Logic 195 is configured to select a request for an action or information from the caregiver, rather than a question to be asked of a user being cared for. As used herein, a “question” is meant to include such requests. For example, an uneven gait may result in questions: “Please observer if Mr. Jones has uneven pupils or has an uneven facial expression or has an uneven grip,” “Please check Mr. Jones for bruises or other signs of a fall,” “Please observe Mr. Jones steadiness while walking” and/or “Please check for empty alcohol bottles.” In some embodiments, a caregiver may be asked to read data from a blood pressure meter, a glucose meter, and/or other medical device. In some embodiments, a care giver may be asked to confirm that the cared for user is taking a medicine or otherwise complying with a care plan.

In some embodiments, Question Logic 195 is configured to select questions based on answers received to prior questions. Such “follow-up” questions may be selected shortly after the answer is received or within a day, a couple of days or a week. For example, a question “have you felt tired or dizzy” is designed to be easier for a person to answer than a question “have you felt dizzy.” And if the user has answered affirmatively to the compound question then the user may be more likely to answer truthfully to a question “was it tired or dizzy or both?” In another example, the question “How did you sleep last night?” and answer “not well” could be followed up by a question “did you get up many times.” The question “Are you having to go to the bathroom lots?” and answer “yes” could be followed up by a question “are you able to pee or does it burn when you pee?” The question “It doesn't look like you did much yesterday, how are you feeling?” and answer “I just feel tired” could be followed up by questions aimed at determining why the user feels tired. For example, “Are you sleeping well?” “Did you eat much yesterday?” or “Do you feel you might be getting a cold?” Sometimes an initial question will be selected to confirm an activity indicated by sensor data and follow-up questions will be directed at learning more information about the activity. For example, a first question may be selected to confirm that the user has increased bathroom trips and follow-up questions are selected to determine a reason for these increased trips.

In some embodiments, Question Logic 195 is configured to confirm a possible determined health state of a user. For example, if a health state of “depressed” is determined as being likely. Questions (e.g., follow-up questions) may be selected to confirm this determination. Answers to such questions may be used by medical professionals to make a medical diagnosis.

In some embodiments, Question Logic 195 is configured to select questions based on a user's know medical history. For example, a user using a catheter, and thus more likely to have infections, may more often be ask questions selected for the early detection of these infections. A user having type II diabetes may receive more questions about diet, exercise and urination. A user suffering from edema may be asked questions about daily walks. Users having post-operative or other recovery plans may be asked questions regarding compliance with these plans. In some embodiments, Question Logic 195 is configured to select questions based on deviation from an expected and/or proscribed activity.

In some embodiments, Question Logic 195 is configured to select questions in order to complete a psychological evaluation. For example, questions can be selected from a standard psychologic test, different questions from the test being asked at different days and/or times. A complete test may, thus be asked over a period of days or weeks. At a time, Question Logic 195 may be configured to select one, two, three or more questions to be ask of a cared for user. The number of questions to be asked, e.g., question budget, may depend on the user's medical (health) state, sensor data, activities, health risk, and/or the like.

Question Logic 195 may be configured to select questions based on value and/or likelihood of improving the performance of Activity Logic and/or Question Logic 195. For example, questions may be selected based on a goal of receiving answers of most value in further training any part of Activity Logic 135 and/or Activity Analysis Logic 730 (discussed elsewhere herein). Question selection may further be dependent on the progress of this training. For example, initially a question may be used to confirm that sensor data represented an eating activity. As confirming answers are received the confidence in the interpretation of this sensor data is increased. The questions used to confirm eating, thus, may become less valuable than questions regarding what was eaten or sleep patterns. Thereby, Question Logic 195 may evolve to select different questions over time. A goal of the evolution of Question Logic 195 is to select more valuable questions as more annotated sensor/activity data is generated, and the various machine learning systems discussed herein are further trained using this annotated data. This training may be applied generally to a cohort and/or to an individual user. Question Logic 195 may be configured to select questions based on balance between a likelihood that the at least one question will result in an answer that provides most value in training a machine learning system and a likelihood that the at least one question will result in an answer that provides most value in determining the health state. The value of a question for a specific goal is optionally based on a sensitivity analysis of questions within the set of questions.

In an illustrative example, radar sensor data may indicate that a user regularly lays on her bedroom floor. This data may initially cause Question Logic 195 to generate questions regarding falls. Resulting answers may identify an unexpected activity including yoga exercises. From these answers, any of the various machine learning systems discussed herein, including Question Logic 195, may be further trained to recognize this activity.

Management Server 123 optionally further includes Polling Logic 188 configured to poll members of Monitored Devices 110. This polling can be used to determine if Monitored Device 110A is operational. The response to a polling request can be considered a use of Monitored Device 110A to be considered by Deviation Logic 160.

Monitored Device 110A and/or Management Server 123 further include a Microprocessor 180. Microprocessor 180 includes a microprocessor, an ASIC, a programmable logic array, a communication circuit, a central processing unit, and/or the like. Microprocessor 180 is typically configured to perform specific tasks by the addition of software and/or firmware. For example, Microprocessor 180 may be configured to execute Activity Logic 135, Reporting Logic 145, Deviation Logic 155, Modeling Logic 192, Alert Logic 186, and/or any of the other logic discussed herein.

Monitoring Device 120 can include any communication device capable of receiving an alert message. In various embodiments Monitoring Device 120 includes a smartphone, personal computing device, e-mail account, and/or the like. Monitoring Device 120 includes a Monitoring Interface 196 configured to receive an alert and present the received alert to a user. The alert can be tactile, audible, and/or visual. In some embodiments, Monitoring Interface 196 is part of a client dashboard available to employees of a care organization. For example, Monitoring Interface 196 may be configured for care managers to view the activities and/or health states of numerous clients.

Monitoring Device 120A optionally includes Alert Response Logic 198 configured to respond to a received alert. Alert Response Logic 198 may be configured to send a text message or place a call to Monitored Device 110A. In some embodiments, Alert Response Logic 198 is configured to automatically connect a call to Monitored Device 110A via Voice Logic 165.

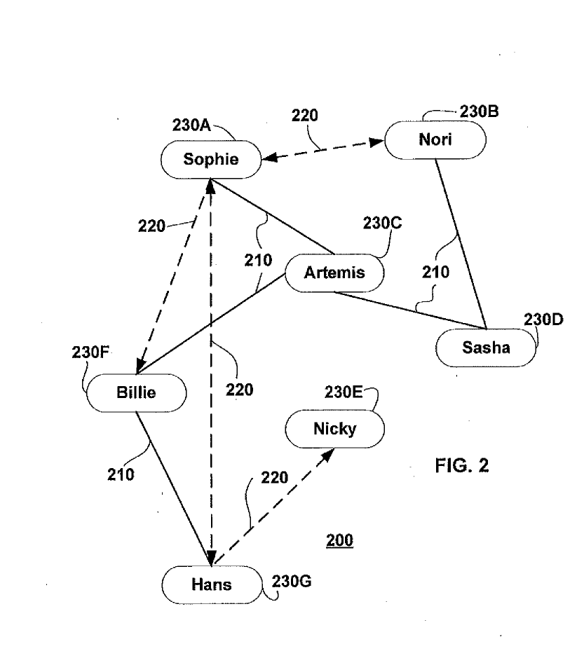

FIG. 2 illustrates a Social Network 200 having more than one type of connection, according to various embodiments if the invention. A Basic Connection 210 is indicated by a solid line and an Enhanced Connection 220 is indicated by a dashed line. Optionally, the Enhanced Connections 220 include all the properties of the Basic Connections 210 and also additional functionality. The members of Social Network 200, individually identified as 230A, 230B, etc., can have Basic Connections 210 in which both members have equal roles (symmetric). Further, they can have Enhanced Connections 220 that are directional, one member being followed and the other member being a follower (asymmetric). The same pair of members can have both types of connections at the same time. In FIG. 2, arrowheads are used on Enhanced Connections 220 to indicate who is following whom. For example, Sophie 230A and Nori 230B are following each other, while Hans 230G is following Nicky 230E, but Nicky 230E is not following Hans 230G. Even while the Enhanced Connection 220 between Hans 230G and Nicky 230E is asymmetric they may simultaneously have a Basic Connection 210 that is symmetric.

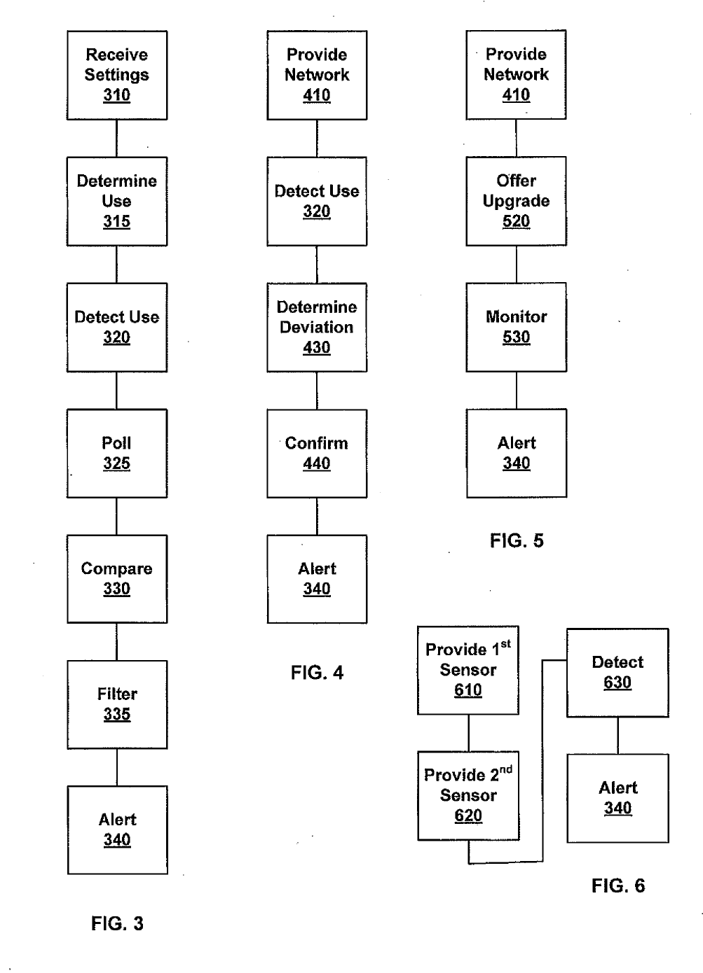

FIG. 3 illustrates methods of passively monitoring a user, according to various embodiments of the invention. In these methods, a user of Monitored Device 110A is monitored and alerts are generated according to an established set of alert rules. These rules can result in an alert as a result of specific actions and/or as a result of a lack of action. The methods illustrated by FIG. 3 may be performed using Monitored Devices 110 and/or Management Server 123.

In an optional Receive Settings Step 310, a set of alert rules and/or privacy criteria are received. These may be received at Monitored Device 110 and/or Management Server 123. The alert rules and/or privacy criteria are used to control issuance of and/or content of alert rules. The received alert rules are may be received from Monitored Device 110A and/or from Monitoring Device 120A. Typically, a user of Monitored Device 110A has an option to approve or disapprove any alert rules and privacy criteria. Receive Settings Step 310 is optional in embodiments wherein a default set of alert rules and/or privacy criteria are established. Receive Settings Step 310 is optionally performed using Setup Logic 155. Different alert rules and privacy criteria are optionally set received for different Monitoring Devices 120A. The received rules and criteria optionally apply to more than one Monitored Device 110 associated with the same user.

As discussed elsewhere herein, the alert rules can relate to specific events, e.g., presence at a hospital or police station, and may relate to a lack of specific events, e.g., a lack of movement or other user of Monitored Device 110A for a period of time. The alert rules and/or privacy criteria are optionally received by using Setup Logic 155 to provide a user interface to Display 125 that is configured to request that a user modify a set of default alert rules and/or privacy criteria. The alert rules and privacy criteria received in Receive Settings Step 310 can include any of those discussed elsewhere herein.

Receive Settings Step 310 optionally further includes receiving identities of Monitored Devices 110 and/or Monitoring Devices 120. For example, a user may designate their cellular phone, iPad, desktop computer, car and/or home security systems as monitored Devices 110. Each of these devices can be associated with one user. For example, a user may designate that their car can report an accident to Management Server 123, that at least one of their cellular phone, iPad and desk computer be used at least once every 16 hours, and/or that their home security system detect movement within their home at least once a day. The alert rules may include logical relationships between these designations. For example, an alert may be sent only if none of the computing devices are used and the home security system doesn't detect any use during the same period. The sending of an alert is, thus, dependent on the use of several monitored devices, which may be separate and independent of each other.

Receive Settings Step 310 optionally further includes receiving privacy criteria from a user of Monitored Device 110A or Monitoring Device 120A. Privacy criteria can include: specific criteria of what information about a user's activity can be sent to third parties. This information may be sent as part of a digest and/or as part of an alert. For example, a privacy policy may specify that location information may not be disclosed unless Monitoring Device 120A is involved in a vehicle accident, found at a police station, found at a hospital, and/or the like. Privacy criteria are optionally dependent on the Monitoring Devices 120 to which the information is being sent. Thus, one may provide more personal information to some followers relative to other followers.

With respect to digests, Receive Settings Step 310 can include privacy criteria for determining what to include in digests and how often digests are to be provided to Monitoring Devices 120. For example, a user may specify that daily digests include a general characterization of how active a user was for the day, e.g., activity ranked on a scale of 1-5. Such a ranking can be calculated by Activity Logic 135 and can be based on, how many steps the user took, use of peripheral devices (e.g., a TV remote), if the user left their house, if the user went to work, and/or the like. Further, the user may specify that a weekly digest includes a graph showing relative activity for each day of the week. A user may provide for adding manually entered comments in a digest, thus the user can add statements such as “I felt great today” or “I'm lonely” to a digest.

In Receive Settings Step 310 a user can optionally designate alert cancellation criteria. These criteria provide users' of Monitored Devices 110 an opportunity to cancel an alert. A user may specify that there should be 3-minute delay if an alert is related to a vehicle accident, a 15-minute delay if an alert is related to presence at a hospital and a 30 min delay if an alert is related to a lack of movement for a period. Typically, cancellation of an alert is accomplished by posting a notice that an alert will occur shortly on Display 125. The notice may indicate the reason for the alert, a time remaining to cancel the alert, and inputs configured to cancel the alert and or send it immediately.

In some embodiments, Receive Settings Step 310 includes receiving data related to a machine-based system configured to characterize use of Monitored Device 110A. For example, Receive Setting Step 310 may include receiving coefficients for an artificial neural network trained to distinguish expected use from deviant use of Monitored Device 110A. These coefficients are optionally specific to the user of Monitored Device 110A, and/or to a group of which the user is a member.

In some embodiments, Receive Settings Step 310 includes identification of Peripheral Devices 172, in direct communication with Monitored Device 110A. Peripheral Devices 172 can be assigned any of the settings, e.g., alert rules and privacy criteria, etc., discussed wherein with respect to Monitored Device 110A.

In a Determine Expected Use Step 315, an expected use of Monitored Device 120A is determined. In a simple implementation, the expected use is merely that the use will not violate alert rules received in Settings Step 310 and/or a default set of alert rules. The expected use can include a location of Monitored Device 110A. For example, the expected use can include that Monitored Device 110A will not be at a hospital, will be at church on Sunday mornings, will leave a home of a user at least once a day, will be present at a school in certain hours, will not leave a defined geographic area, and/or the like. The expected use can include a movement of the Monitored Device 110A or a Peripheral Device 172 connected to Monitored Device 110A. For example, the expected use can include not traveling at a speed greater than 75 mph, walking at least 5000 steps per day, being picked up at least once in a predetermined time period, opening of a door, flushing of a toilet, getting out of bed as determined by a pressure sensor, activation of a pressure sensor or motion detector, and/or the like. In some embodiments, the expected use includes use of Internet of Things (IoT) devices, use of a vehicle (e.g., car or motorized wheelchair), and/or use of a security system.

The expected use can take into consideration multiple devices. For example, in may be expected that the user uses at least one of a vehicle, a tablet computer, a cell phone or home motion sensors at least once every 12 hours. If just one of these devices is used, then the expected use is satisfied.

In a Detect Use Step 320, the use of Monitored Device 110A is detected. The use may be detected in a variety of ways. For example, if Monitored Device 110A is a mobile device, e.g., a smart phone or tablet computer or vehicle, then the detected use may be detected by receiving a signal from Motion Sensor 140, a gyroscope, accelerometer, or Global Positioning System (GPS). The use may be detected by detecting use of Display 125 and/or a specific application on Monitored Device 110A. The detected use of a tablet computer or smartphone may include merely picking up the device.

The use detected in Detect Use Step 320 can include detection of IoT devices or a security system. For example, opening of a smart refrigerator door, use of a bathroom, adjustment of a thermostat, use of a remote control (e.g., a TV remote), opening of a door or window as detected by a security system, placing or removing weight from a pressure sensor, sue of a dishwasher, use of a washer/dryer, noise detected by a microphone, motion detected by an infrared motion detector, motion detected by a camera, use of a mechanical bed, turning on/off lights or other appliances, use of a CO sensor, use of a smoke/fire sensor, use of a stove or oven, use of a toothbrush, use of a door lock, use of a firearm, use of a heater or air conditioner, use of a temperature sensor, use of electricity, use of a coffee maker, use of a toaster, use of a mechanical chair, use of a television, use of an audio system, use of a wearable device, use of a battery charging device, use of a personal computer, and/or the like.

The use detected in Detect Use Step 320 optionally includes detecting use of multiple Monitored Devices 110 and/or of Peripheral 172. For example, the detected use may include use of an Apple Watch™, a Fitbit™, a glucose sensor, a heart sensor, an insulin pump, a pacemaker, an automatic defibrillation device, a pulse monitor, electronic glasses, a prosthetic, an oxygen sensor, a hearing aide, and/or the like. More specifically, the detected use may include determining that a user is asleep at night based on a heart sensor and that upon waking they use the restroom, turn on the TV and check their messages on their smartphone.

Note that the use detected in Detect Use Step 320 optionally includes a lack of use.

In an optional Poll Step 325, Management Server 123 sends message to Monitored Device 110A and receives a response if Monitored Device 110A is on and functional, e.g., awake. Lack of response for a designated period is optionally grounds for sending an alert. Poll Step 325 is optionally performed using Poll Logic 188.

In a Compare Step 330, the use detected in Detect Use Step 320 is compared with the expected use as determined in Determine Use Step 315. This comparison may be performed by Activity Logic 135 and may be performed on either Monitored Device 110A or Management Server 123. If the detected use deviates from the expected use, then an alert may be sent to Monitoring Device 120A. In one example, the expected use is determined in part by alert rules set in Receive Setting Step 310. These alert rules include that the user use at least one of their television, smartphone, personal computer or tablet computer at least once a day. If the detected use does not include use of any of these devices for a day, then an alert may be sent.

In an optional Filter Step 335, the use as detected in Detect Use Step 320 is filtered using Filter Logic 150. This filtering is optionally performed according to privacy criteria set in Receive Settings 310. The filtering optionally results in creation of a summary of detected use, e.g., a digest of the detected use. The detected use may be filtered on Monitored Device 110A and/or on Management Server 123 (using an embodiment of Filter Logic 150 included therein). Filtering of the detected use an improve privacy.

Filtering of the detected use is optionally dependent on a destination of an alert or a digest. For example, a user may have indicated that a greater amount of information be sent to Monitoring Device 120A relative to Monitoring Device 120B.

In an illustrative embodiment, prior to sending an alert, Filter Step 335 includes removal of all detected use that isn't relevant to the alert. Specifically, if the alert is a result of Monitored Device 110A visiting a hospital or being in a car accident, then the location of the hospital or accident may be included in the alert, but additional information as to where a user has traveled is removed. Prior to sending a digest, Filter Step 335 may include removing information as determined by privacy criteria. This information may be summarized, e.g., stating merely that the user was “inactive,” “somewhat active,” “active” or “very active” based on the amount of activity detected.

In an Alert Step 340, an alert is sent to one or more of Monitoring Devices 120. The alert may be sent from Monitored Device 110A or from Management Server 123. The alert is based on the comparison of Compare Step 330 and a determination therein that an alert rule was violated and/or that the actual use deviated from the expected use. Alert Step 340 is optionally performed using Alert Logic 186.

The alert may be sent to a specialty application executing on Monitoring Device 120A, e.g., to an application supporting Monitoring Interface 196. Alternatively, the alert may be sent as a voice call, a text message or e-mail. In some embodiments, Alert Step 340 includes providing the user of Monitored Device 110A an opportunity to cancel the alert or add information to the alert. For example, this user may provide text indicating that she is only at the hospital to visit the pharmacy.

Typically, the alert includes an explanation as to why the alert is being sent. For example, that Monitored Devices 110A has not been moved, that a person has not gotten out of bed, that a person is in unusual location, a person has only walked 300 steps in a day, and/or the like. An Alert may be sent to more than one Monitoring Devices 120.

In some embodiments, Alert Step 340 includes sending a response to the alert from Monitoring Device 120A to Monitored Device 110A. This response may be initiated using Monitoring Interface 196 and/or may use Voice Logic 165 to open a voice channel. The response may include text message(s), voice communication, and/or the like.

FIG. 4 illustrates methods of managing a social network, according to various embodiments of the invention. In these methods, a social network includes two types of connections, referred to herein as “basic” and “enhanced” connections. The enhanced connection including automatic monitoring of use of Monitored Devices 110. The monitoring including generation of alerts if use of the Monitored Devices 110 deviates from an expected use and/or violates alert rules set by users of the Monitored Devices 110. Typically, the basic connection does not include automatic monitoring of the use of the mobile device of the first user.

In a Provide Network Step 410, a social network is provided to multiple members. The provided social network includes basic connections between some members and enhanced connections between a subset of those members having basic connections. The enhanced connections are optionally one-way, e.g., between a follower and person being followed. The basic connections may be like those found in Facebook™, LinkedIn™, Snapchat™ and Instagram™. Provide Network Step 410 optionally includes an embodiment of Receive settings Step 310 for each network member that is part of an enhanced connection, or a followed member of the enhanced connection.

In an embodiment of Detect Use Step 320, discussed elsewhere herein, the use of Monitored Devices 110 is detected. This monitoring is optionally only performed for those members of Monitored Devices 110 associated with network members that are followed members of an enhanced connection, e.g., for Monitored Device 110A.

In a Determine Deviation Step 430, it is determined that the detected use of Monitored Device 110A is outside of the expected use and/or alert rules are violated. Determine Deviation Step 430 optionally includes Determine Use Step 315, Detect Use Step 320, Poll Step 325, and/or Compare Step 330.

In an optional Confirm Step 440, the user of Monitored Device 110A is provided with an opportunity to cancel automatic delivery of the alert to a second member of the social network, e.g., to a follower of the user of Monitored Device 110A who is associated with Monitoring Device 120A.

If the second member does not take assertive action to cancel the alert within a predetermined time period, the alert is sent to Monitoring Device 120A in an embodiment of Alert Step 340. As discussed elsewhere herein, in Alert Step 340 an alert is provided to a second user associated with Monitoring Device 120A. The alert indicates that the detected use of Monitored Device 110A has deviated from the expected use. The provision of the alert to the second user is based on an enhanced connection between the first user and the second user.

FIG. 5 illustrates methods of upgrading a social network, according to various embodiments of the invention. In these methods, several enhanced connections relative to basic connections in a social network are increased. For example, a social network having 100 basic connections and 0, 2 or 10 enhanced connections, may be upgraded to include at least 15 or 20 enhanced connections for every 100 basic connections. In some embodiments, a user may pay for enhanced connections.

In Provide Network Step 410, the data representing a social network including multiple members is received. This data may include information characterizing members of the network and which members have basic connections with each other. The social network optionally further including features that allow text messaging and sharing of content (e.g., videos, images or links) between the members. Each of the members of the social network has a set of basic connections to one or more other members of the social network.

In Offer Upgrade Step 520, one or more members of the social network are provided an upgrade opportunity. The upgrade opportunity includes an ability to establish an enhanced connection between the first of the members and a second of the members of the social network. The enhanced connection includes automatic monitoring of use of a mobile device, e.g., of Monitored Deice 110A, of the second of the members and reporting of the automatic monitoring to the first of the members, e.g., reporting to Monitoring Device 120A. The automatic monitoring includes detection of use that violates alert rules and/or deviates from an expected use of Monitored Device 110A. This excludes expected use such as an acknowledgement that a user has read a text massage, that Monitored Device 110A is low on power, or that a user is in an expected location.

Optionally, the automatic monitoring includes detection of movement of the mobile device using an accelerometer or gyroscope. This movement can be as small is picking up or rotating the mobile device.

Optionally the enhanced connection is enhanced relative to a basic connection between the first and second members, the basic connection not including automatic monitoring of user of the mobile device of the second of the members.

In a Monitor Step 530, the use of Monitored Device 110A is monitored using the methods and/or systems discussed elsewhere herein. For example, using the methods illustrated in FIG. 3.

In an embodiment of Alert Step 340, an alert is sent to Monitoring Device 120A based on the monitoring. The alert is sent if the use of Monitored Device 110A deviates from and expected use and/or violates an alert rule agreed to by the user of Monitored Device 110A. As noted elsewhere herein, the alert is optionally based on a lack of use includes a lack of movement of Monitored Device 110A. The alert is optionally based on receiving a report that the mobile device has not been used for a period and automatically reporting the lack of use to monitoring Device 120A.

FIG. 6 illustrates methods of monitoring a person's activity using multiple sensors, according to various embodiments of the invention. In these methods, the outputs of the multiple sensors are used to determine if an alert should be sent. Some alert rules are dependent on the output of either sensor, some alert rules are dependent on the outputs of one sensor but not others, some alert rules are dependent on multiple sensor outputs.

For example, if a first sensor is an accelerometer within a smartphone, then one alert rule may be that an alert should be sent if the smartphone undergoes a deceleration indicative of a car accident. This alert rule is optionally dependent on the output of just the first sensor. In another example, if the first sensor is an accelerometer in a smartphone and a second sensor is a pressure pad under a carpet. Then an alert rule may specify that if the smartphone isn't moved and the pressure pad does not detect a step for a period, then an alert should be sent.

In a Provide 1^{st} Sensor Step 610, a first sensor having a first area of regard is provided. In a Provide 2^{nd} Sensor Step 620, a second sensor having a second area of regard is provided. The first and second sensors may either or both be motion sensors. The first and second sensor may include any combination of the sensors discussed elsewhere herein. In some embodiments, both the first sensor and the second sensor are both part of a home security system.

In a Detect Step 630, outputs of the first and second sensors are considered. For example, in some embodiments, it is determined that motion has not been detected by either the first or second sensor for a predetermined period. This may mean that the user of Monitored Device 110A has neither picked up their phone nor walked down the hall to the bathroom in the time period under consideration. Alert rules optionally specify that Boolean operations be applied to the outputs of the first and second sensors in order to determine if an alert should be sent.

In Alert Step 340, an alert is provided to a remote destination, e.g., Monitoring Device 120A. The alert may indicate a lack of detected motion by both or either the first and second motion sensor. The alert is optionally sent to a plurality of Monitoring Devices 120. Typically, the Monitoring Devices 120 to which the alert is sent are associated with followers of a person associated with Monitored Device 110A.

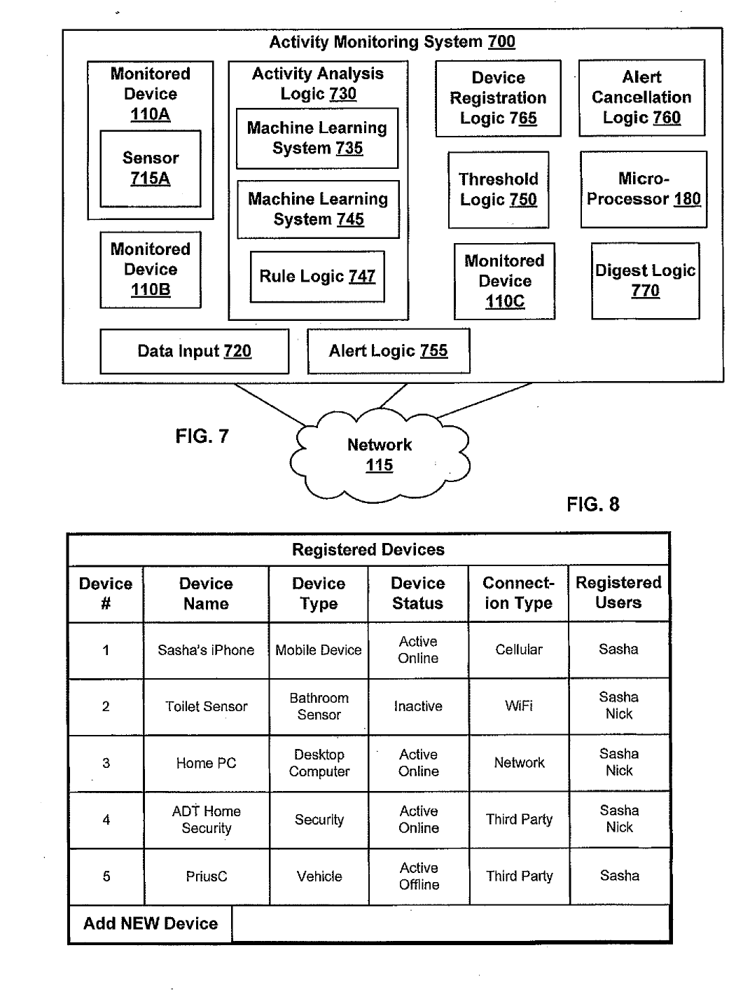

FIG. 7 illustrates a sensor-based Activity Monitoring System 700, according to various embodiments of the invention. Activity Monitoring System 700 is optionally an embodiment of Monitoring System 100, or included in an embodiment thereof, and vice versa. For example, Activity Monitoring System 700 can include multiple Monitored Devices 110, multiple Peripherals 172 and any of the elements described as being included in Management Server 123 (some of which are illustrated in FIG. 7). In these embodiments, a variety of different sensors are used to detect the activity of a user. These sensors may be embodied in different types of devices. Data received from the sensors are optionally used in combination. For example, an unexpected lack of activity may be determined using a combination of sensors in a smartphone, a personal computer and motion detectors that are part of a home security system. The lack of activity is determined based on a lack of detected activity in any of the devices. Thus, even if no activity is detected on the smartphone, the user is considered active if activity is detected on the home security system. Activity Monitoring System 700 can include a mobile device, a cloud-based computing system, a server, and/or distributed set of computing devices.

As used herein, “different types of devices” are devices of different primary functionality or different form factor. For example, smartphones having different operating systems would both be considered devices of the same type, but a smartphone and a tablet computer would be considered different devices because the tablet computer does not include the primary functionality of communications via a cellular network. A tablet computer and a laptop computer would be considered different types of devices because of their different form factors. An indoor radar and an infrared motion detector would be considered different types of devices because of the different types of data they generate. Further examples of different types of devices include wearable devices, TV remotes, video display devices, internet TV devices, smartphones, home security components, vehicles, thermostats, IoT appliances, heart beat sensing devices, glucose sensing devices, blood pressure sensing devices, a motion detector, a radar, a security device, a bed pressure detector, a toilet use detector, an entry detection device, a vehicle, a medical device, a thermostat, a personal assistant, a television or television remote, a microwave, a stove, a refrigerator, a tablet computer, a personal computer, a toothbrush, a coffee maker, a smoke detector, and/or the like.

Activity Monitoring System 700 includes multiple Monitored Devices 110, individually referenced herein as Monitored Device 110A, 110B, 110C, etc. Monitored Devices 110 can include any combination of the various devices discussed herein. As discussed, Monitored Devices 110 are configured to monitor activity of a user using multiple Sensors 715, such as Motion Sensor 140 and/or Peripheral 172. Different types of Monitored Devices 110 and Sensors 715 can detect different types of activity. For example, a smartphone may use motion sensors that detect motion of the smartphone to detect several steps a person takes or the evenness of their gait. A medical device may detect glucose levels, electrical signals within a user's body, heart rate, and/or the like. Pressure sensors may detect movement within a house, a user lying in bed or use of a toilet. Indoor radar can detect movement, gait, breathing, and in some cases distinguish between people having different size, walking pace, movement patterns, etc. Each of Monitored Devices 110 includes at least one of Sensors 715, individually referenced as 715A, 715B, etc.

Sensors 715 may detect different physical phenomena and transduce these to electrical signals. Sensors 715 my optionally further digitize and communicate the electrical signals to other parts of Activity Monitoring System 100. Sensors 715 may detect pressure, motion, orientation, chemicals, temperature, mass, strain, sound, light, voltage, current, location, acceleration, physiological conditions, shape, size, breathing, and/or the like. Sensors 715 may include a keyboard or touch screen.

Activity Monitoring Systems 700 further includes Data Input 720 configured to receive the electrical signals from Sensors 715. As Monitored Devices 110 can include a wide variety of devices, these devices may communicate to Data Input 720 via a variety of different communication channels. For example, communication between Monitored Device 110B and Data Input 720 may occur through a Network 115. In one illustrative example, Monitored Device 110A is a smartphone that communicates data using a cellular data network; Monitored Device 110B is a tablet computer that communicates data using a virtual private network connected to the internet; and Monitored Device 110C is a home security system that communicates data via a Monitored Device 110B. The data communicated can include raw sensor data, partially processed sensor data, determined activities, and/or conclusions generated from the analysis of sensor data. Data Input 720 can include modems, gateways, firewalls, serial ports, Ethernet ports, radio frequency antennas, and/or computing instructions configured to receive data generated using or derived from Sensors 715.

Activity Monitoring System 700 further includes Activity Analysis Logic 730. Activity Analysis Logic 730 is optionally an embodiment of Activity Logic 135 and/or Modeling Logic 192. Activity Analysis Logic 730 is configured to determine an activity level of a user based on data generated using Sensors 715 and optionally received via Data Input 720. Activity Analysis Logic 730 may further be configured to determine a health state of the user based on the activity level. Activity Analysis Logic 730 can include a first Machine Learning System 735, a second Machine Learning System 745, and/or Rule Logic 747. Parts of Activity Analysis Logic can be disposed within Monitored Devices 110, within Management Server 123, or distributed between Monitored Device 110A and Management Server 123. For example, part of Activity Analysis Logic 730 may be disposed as an application on a smartphone or tablet computer. This part of Activity Analysis Logic 730 may be used to perform initial processing of data generated using Sensors 715, the output of this initial processing may then be sent via Network 115 to other parts of Activity Analysis Logic 730 located on Management Server 123 for further processing.

Rule Logic 747 includes a rule-based logic configured for determining an activity from sensor data and/or configured for determining a health state from an activity (or deviation from expected activity). Rule Logic 717 can include an “expert system,” a Bayesian model or Bayesian statistical system, rules directly coded in computing instructions, statistical analysis logic, probability models, and/or the like. Rule Logic 747 is a machine-based system but not a machine learning system. While the accuracy of Rule Logic 747 can improve statistically with the availability of additional data, Rule Logic 747 is not “trained.”

Activity Analysis Logic 730 is configured to detect changes in a person's activity. Changes in a person's activity can be suggestive of a wide variety of medical conditions. These changes may occur over time periods of at least one hour, at least one day, at least one week, or at least one month. By way of example:

a) depression may be indicated by isolation (not going out), the use of online social networks (rather than face-to-face interaction), changes in sleep patterns; and/or lack of physical activity;

b) sleep disorders may be indicated by frequent waking and irregular sleep patterns;

c) digestive disorders may be indicated by frequent visits to the toilet;

d) diabetes may be indicated by frequent urination or getting up several times during the night;

e) mental illness (of which there are many varieties) may be indicated by erratic or repetitive behavior, by dramatic changes in activity levels or by changes in social patterns;

f) arthritis may be indicated by changes in physical activity, for example, typing or walking speed;

g) cardiac distress may be indicated by a reduction in a person's ability to walk a distance without breaks or to climb stairs at a steady pace;

h) anemia may be indicated by a decline in general physical activity;

i) alcoholism may be indicated by visits to locations selling alcohol, by frequent occurrence of an unsteady erratic gait or by driving patterns;

j) memory loss may be indicated by an inability to remember passwords, by becoming lost or by forgetting keys or a smartphone;

k) stroke may by indicated by a sudden change in gait, loss of balance or by differences in typing speed between the right and left hand, imbalance in eye, dilation or facial expression;

l) vision loss may be indicated by changes in display font size or frequent movement of a smartphone toward and away from the eyes;

m) loss of joint function (e.g., hip) may be indicated by changes in walking speed, uneven gait or reduced physical activity;

n) physical therapy progress may be indicated by range of motion;

o) compliance with advice to elevate a limb may be indicated by limb position;

p) asthma may be indicated by a shortening of periods of physical activity;

q) allergy may be indicated by seasonal changes in physical activity;

r) colds and flu may be indicated by a reduction in physical activity and extended time spent in bed or at home;

s) fatigue and low energy may be indicated by a decline in physical activity;

t) hearing loss may be indicated by a gradual increase in volume settings on electronic devices;

u) kidney disease may be indicated by changes in urination patterns;

v) Parkinson's disease may be indicated by muscle tremors;

w) dementia may be indicated by a reduction in ability to remember things (e.g., passwords), changes in typing speed, getting lost, or changing in driving patterns;

x) stress may be indicated by changes in sleep patterns and physical activity; and/or

y) cancer may be indicated by activity related to the impacted organ or organs, for example Pancreatic Cancer may be indicated by a decrease in appetite, brain cancers may have indications like stroke, but with slower onset, leukemia may have indications similar to anemia.

z) Bowel or urinary problems may be indicated by time spent on a toilet and/or frequency of bathroom visits.

Activity Analysis Logic 730 can be configured to detect these and many other activity changes that may be indicative of health states, including medical problems warranting medical care.

Activity Analysis Logic 730 is optionally configured to determine the activity level of a user based on a set of rules, embodied in Rule Logic 747 or other machine-based system. For example, if the user's smartphone is determined to be at a hospital using GPS sensors, then the activity level of the user may be determined to include visiting a hospital. In another example, the activity level of the user may be based on use of a TV remote, on use of a computer keyboard, or on use of a specific software application/website.

Activity Analysis Logic 730 optionally includes one or more machine learning system, such as Machine Learning System 735 or Machine Learning System 745. Machine Learning System 735 is configured to derive the activity level of the user, from sensor data, based on a trained neural network or other trained expert system. Machine Learning System 735 is configured to receive data resulting from Sensors 715 to determine the activity level. For example, Machine Learning System 735 may be trained to determine that a specific pattern of acceleration of a smartphone is indicative of a car crash, that a pattern of motion can be used to determine a number of steps taken by a user, that a pattern of typing on a keyboard can be indicative of a stroke, that an uneven gait can indicate a hip problem, alcohol consumption or a stroke, that a specific pattern of acceleration can indicate a cough or sneeze, and/or any of the other activity conditions discussed herein.

All or part of Machine Learning System 735 is optionally disposed on members of Monitored Devices 110. For example, acceleration data generated by Sensor 715A on Monitored Device 110A may be processed by Machine Learning System 735 to produce a preliminary result indicative of an activity, this preliminary result on communicated from Monitored Device 110A to other parts of Activity Analysis Logic 730 (e.g., via Data Input 720 and/or Network 115) only if the preliminary result is indicative of a car accident, presence at a hospital or police station, and/or other specific activity of concern. Thus, sensor data may first be analyzed on Monitored Device 110A and then further analyzed at a remote location including elements of Activity Monitoring System 100. The remote location can include a server including a Microprocessor 180 configured to execute any of the various logic discussed herein. Machine Learning System 735, Machine Learning System 745, and/or Rule Logic are optionally configured to analyze images or a series of images. For example, Machine Learning System 735 may be configured to analyze an image of a wound for signs of infection or to analyze one or more images for indications of skin cancer, and/or other uses of image analysis discussed herein.

Activity Analysis Logic 730, or specifically Machine Learning System 735, are optionally configured to use data received from multiple Monitored Devices 110 to determine an activity level of a user. For example, lack of activity detected at multiple Monitored Devices 110 may be used to determine a low level of activity of a user. Different Monitored Devices 110 are optionally associated with different users. For example, in a household in which two users live the home security system and the TV remote may be associated with both users, while personal cell phones or tablet computers may be separately associated with the different users. In such embodiments, Activity Analysis Logic 730 may be configured for associating different activity data with the different users. Some activity data (e.g., a motion or door sensor that is part of a home security system) may be associated with more than one user.

In some embodiments, Activity Analysis Logic 730 is configured to use data from multiple sensors to classify data from a specific sensor as being associated with a user at a location. For example, if two users occupy a house monitored using indoor radar or an infrared sensor and these sensors detect movement, then temporal correlation with a wearable sensor may be used to identity which of the two users is moving as detected by the radar or infrared sensor. Specifically, the user whose wearable detects movement may be assumed to be the user whose movement is detected by the non-wearable (stationary) sensor.

In some embodiments, Activity Analysis Logic 730 is configured to treat sensor data generated during a caregiver visit differently from sensor data generated at times when caregivers are absent. This distinction can be used to avoid having activities of the caregiver confused for activities of the user. For example, if a caregiver visits the bathroom, does laundry and cooks a meal, then these activities are preferably not classified as being activities of a senior or other user, for which the caregiver is providing services. Rather, they may be classified by Activity Analysis Logic 730 to create an audit trail as to services performed for the user.

In a specific example, when a caregiver arrives at a user's residence, the caregiver may begin charging of the user's wearable device (e.g., Peripheral 172). The caregiver may then begin a list of service tasks to be performed on behalf of the user. This list of services may be represented by a care plan and can include items such as changing sheets, bathing the user, feeding the user, or taking the user for a walk. The motion detected as the result of these activities can be used to verify that the caregiver completed these items in the care plan.

Times when a caregiver is present can be determined in a variety of ways. In some embodiments, a caregiver may check in and out of their shift using Caregiver Interface 158. This can be done, for example, with a username/password, barcode, and/or detecting of a wireless device in the possession of the caregiver. In some embodiments, the caregiver carries an RFID device or a wearable device that can be detected by Monitored Devices 110. Movement detected by the caregiver's wearable device (e.g., and embodiment of Peripheral 172) may also be used, by Activity Analysis Logic 730, to confirm that specific activities were performed by the caregiver. For example, if a list of services to be provided by the caregiver including doing laundry, then the caregiver should be detected moving near the laundry room. The various elements of Monitored Devices 110 and Management Server 123 may, thus, be used to generate an audit trail of services provided by the caregiver.

Activity Analysis Logic 730 or Activity Logic 135 are optionally further configured to determine an expected activity level for a user. The expected activity level may be based on demographics of the user and/or actual activity of the user measured using one or more of Monitored Devices 110. For example, the expected activity may be based on the user's age, profession, gender, residence location, health history, etc. Specifically, if the user is a nurse, then visits to a hospital may be considered part of an expected activity level for that user. If the user is a college student, then at least 5000 steps per day may be considered part of an expected activity level for that user.

The determination of an expected activity level of a user based on actual (detected) activity of the user, may be dependent on how long the user has been monitored using Activity Monitoring System 100. For example, when the user is first monitored, a lack of sensor data may make such a determination unreliable. As more data representative of the user's actual activity is collected, the calculation of expected activity from actual activity is expected to become more reliable. The expected activity level of a user is optionally further based on answers received to questions selected using Question Logic 195. For example, if a user says they plan to exercise more, then an expected number of steps taken may be increased. In some embodiments, Activity Expectation Logic Activity Logic 135 or Activity Analysis Logic 730 is configured to first determine an expected activity of the user based on demographics of the user, and then to further refine the expected activity based on actual activity of the user as determined by Activity Analysis Logic 1730 based on data from Sensors 715, and/or answers to selected questions. In these cases, the expected activity is based on both demographics and measured activity. The relative weight of the dependencies on these two sources may change over time. For example, as confidence that measured activity of a user represents future activity increases, the measured activity may be weighted more highly relative to the historical activity for a specific demographic (e.g., cohort of users).

The activity level of a user (expected or actual) can be a multi-dimensional or multi-component parameter. For example, the activity level can include a number of steps taken, time spent in bed, time spent at home, locations visited, toilet or appliance use, number of falls, use of specific software applications (e.g. Facebook), time spent at work, number of coughs, medication taken, heart rate range, walking speed, glucose levels, and/or the like.

Activity Analysis Logic 370 optionally includes two distinct machine-based systems. For example, a trained machine learning system (Machine Learning System 735) may be used to process sensor data and output one or more specific activity based on the sensor data. For example, radar data may be processed to determine sleep quality, gait, respirations, and/or other activities discussed herein. A second part of Activity Analysis Logic 730 (Machine Learning System 745) may be configured to then process one or more specific activities determined by Machine Learning System 735 to determine a specific (actionable) health state and/or a deviation from expected activity. For example, a sudden change in gait may be used to determine that a user should see a doctor for evaluation for a knee injury, stroke, and/or alcohol consumption. The first and second parts of Activity Analysis Logic 730 optionally include separate neural networks (e.g., separately trained deep learning systems). Alternatively, the first and second parts may be combined into a single machine learning system that is trained as a single unit. The single machine learning system being capable of receiving sensor data and outputting a health state therefrom.

Activity Analysis Logic 730 optionally includes Machine Learning System 745. Machine Learning System 745 is a “machine-based system” as defined elsewhere herein and is optionally part of Deviation Logic 160 and/or Modeling Logic 192. Machine Learning System 745 is configured to receive measured/determined activity of a user and based on the received activity determine possible health states for the user. Machine Learning System 745 can be trained based on the measured activities of multiple users. Machine Learning System 745 optionally includes a neural network that embodies an expected activity for the user. Machine Learning System 745 optionally includes embodiments of Modeling Logic 192 and Vice Versa. While Modeling Logic 192 may be configured to detect differences between expected and detected activity of a user, Machine Learning System 745 is configured to determine health states (or probabilities thereof) for a user based on detected activities of the user. In various embodiments, combinations of these approaches may be used to manage care, send alerts. and/or determine health states. The range of expected activities or health states is optionally represented by probability distributions over multiple dimensions.

In addition to determined activities, Machine Learning System 745 may also take as input annotations regarding the sensor data or activity as provided by a caregiver. For example, sensor data and/or a determined activity may be correlated with annotations solicited using questions provided to a caregiver by Question Logic 195. These annotations may be received via Caregiver Interface 158 and Network 115. The annotations can be considered as “ground truth” as to a health state represented by the activity of the user or may be used to further define or clarify the activity. For example, if movement data indicates that the user had frequent bathroom visits in the last 24 hours, then the textual or audio annotation may include an answer “yes” to a question “do you feel like you have to go to the bathroom lots?” This answer is confirmation that the detected activity is bathroom visits. Alternatively, an answer “No, but the toilet keeps running” would indicate a different activity. In this case the annotation is being used to verify “ground truth” for the activity. In another example, an answer of “yes, but I have not been able to poop” both confirms the activity of visiting and further defines the activity. This further definition may be used, by either a medical professional or a machine-based system, to distinguish between constipation and a urinary tract infection.

In some embodiments, Activity Analysis Logic 730 includes Machine Learning System 735 and Rule Logic 747. In these embodiments, Machine Learning System 735 may be configured to determine a user activity based on sensor data, while Rule Logic 747 is configured to suggest possible health states based on the determined user activity. As discussed elsewhere herein, dependence on Rule Logic 747 to determine a health state may change over time. The second part of Activity Analysis Logic 730, e.g., Machine Learning System 745, may be used in parallel with, or alternatively to, Rule Logic 747 to make this determination. As accuracy of Machine Learning System 745 for determining health state and/or deviation from expected activity improves over time, dependent on Rule Logic 747 may be reduced.

In summary, the analysis of sensor data to determine a health state can be a one or two step process. In the one step process, a single machine learning system receives sensor data and determines a health state, which is provided as output. Alternatively, in the first step of a two-step process, Machine Learning System 735 is used to interpret sensor data and determine one or more activities based on the sensor data. In a second step, the one or more activities are used to determine a health state of a user. The second step may be accomplished using Rule Logic 747 and/or Machine Learning System 745.

For example, in the two-step process, output of the first step could include activities “uneven gait,” “restroom visit,” “sleep,” “fall,” “open refrigerator,” “steps taken,” and/or the like. Machine Learning System 745 and/or Rule Logic 747 would receive the activity(ies) and determine a health state therefrom. The input to the second step would be a stream of activity events and the output would be one of several possible health states. The health states could include “frequent restroom visits,” “urinary tract infection,” “gait has become more uneven,” “sudden change in gait,” “not eating,” etc. These may be used by Alert Logic 755 to generate an alert to be communicated to a caregiver or family member, etc.

An advantage of the two-step approach is that the sensor analysis is somewhat generic to all users but the determination of a health state from activities can be individualized. Further, in the two-step approach, the second step may use Rule Logic 747 in addition to or as an alternative to a machine learning system, such as Machine Learning System 745. Further, in the two-step process, a determined activity may be used by Question Logic 195 to solicit annotation from a caregiver (or user). As described elsewhere herein, an answer to the solicitation may be used as “ground truth” regarding the activity or to further define the activity. The annotation may be used in the second step of the two-step process, thus improving the quality/accuracy/value of the determined health state. Further the annotation may be used to further train parts of Activity Analysis Logic 730.

As such, Activity Monitoring System 100 can include multiple machine learning systems, optionally implemented on the same computing device(s). Machine Learning System 735 is trained to determine a measured activity based on sensor data and Machine Learning System 745 is trained to determine a health state or an expected activity based on the measured activity. These determinations may be made for one or more specific users. Both machine learning systems are optionally configured to perform pattern recognition and temporal analysis on their input data.

Activity Monitoring System 100 further includes Threshold Logic 750. Threshold Logic 750 is configured to determine if a difference between expected activity of a user and measured actual activity of the user is greater than a threshold. Alternatively, Threshold Logic 750 may be configured to determine a confidence in a determined health state. Threshold Logic 750 is optionally part of Deviation Logic 160. The threshold may be different for different dimensions of the activity level of the user. For example, presence at a police station or hospital may be compared using a simple yes/no threshold, while several daily steps taken, or a heart rate may have a threshold based on a fixed value or a probability. For example, several steps taken during a day may have a threshold set to be exceeded with less than a 2% probability based on a probability distribution of an expected user activity level.

Thresholds or confidences used by Threshold Logic 750 are optionally time dependent. For example, a lower level of activity may be expected at night when a user is sleeping, or during weekdays when the user spends time working at a desk, relative to weekend days when the user regularly goes mountain biking.

Thresholds used by Threshold Logic 750 are optionally dependent on a predicted accuracy of the expected activity level of the user or a predicted accuracy of a determined health state. For example, if the threshold is based only on demographic information about the user, then the threshold may be set higher, relative to a case in which a substantial amount of actual user activity has been determined using Monitored Devices 110. As such, threshold(s) may be reduced as more data is collected about a user actual activity levels based on data collected at Monitored Devices 110. An additional machine learning system (not shown) is optionally trained to determine dynamic thresholds. This machine learning system may be trained using activity data collected from multiple users using Activity Monitoring System 100. The threshold(s) may be adjusted (for one or more dimensions) in order to achieve a desired false positive and/or false negative rate. Thresholds may also consider changes in more than one dimension. For example, if driving patterns, typing patterns and gait all change in a way that could be indicative of a stroke, then the correlation between these changes may justify use of a lower threshold, relative to thresholds for each of the individual changes.

Activity Monitoring System 100 further includes an Alert Logic 755 configured to send an alert to one or more followers of a user, e.g., family member, caregiver or caregiver agency. Alert Logic 755 is optionally an embodiment of Alert Logic 186. As discussed elsewhere herein, an alert is sent if one or more dimensions of the (measured) activity level of the user are sufficiently different than the expected activity level. Alternatively, an alert may be sent if confidence in a determined adverse health state is above a threshold. The alert may be sent via text message, multi-media message, e-mail, telephone call, caregiver dashboard, and/or any other communication system. Alerts are optionally sent via Network 115. Typically, the sent alert includes an indication of why the alert was sent. For example, an alert may state that the user has coughed repeatedly for 6 hours, has not used to toilet for 10 hours, or has not gotten out of bed by 11 AM, has stroke symptoms, has not left the house in 4 days, has (listed) symptoms of a stroke, and/or the like. An alert may be generated for acute events that happen in a short time or for events that develop over time. For example, an alert may be generated if it is observed that a user's blood glucose has been progressively unstable over several days, that a user's walking distance each day has slowly dropped over a month, and/or that the user's activity level has changed over several weeks in a way that indicates depression. Alerts may include a location of Monitored Device 110A. For example, if an alert is based on presence at a hospital or on data suggestive of a car accident, the alert may include a geographic location. The alert may include detected activities of the cared for user.

Alerts typically suggest that the user receive medical attention rather than providing a medical diagnosis. Once such activity levels (or changes) or health state are detected, that may indicate a problem, the user, and optionally their followers, are advised to seek advice of a medical professional who can make a medical diagnosis. Detection of specific conditions can be more precise if the data is generated by an IoT enabled medical devices. For example, an insulin pump or an inhaler can detect a specific physiological condition or distress. Activity Analysis Logic 730 is typically configured to detect both acute events such as a car crash or fall, and medically relevant events that take place overtime. For example, some of the conditions discussed above are indicated by changes in a person's activity over time.

In some embodiments Alert Logic 755 is further configured to periodically send a digest to followers of the user. For example, Alert Logic 186 may send a daily digest that includes a high-level summary of the activity level of a user. The digest may include a graph representing daily activity level. This representation can be based on multiple factors, such as trips out of the house, number of steps taken, meals cooked, appetite, exercise, etc. In some embodiments, detailed information regarding a user's activity, such as their exact location or where they went is excluded from digests to preserver privacy of the user.

The followers of a user are typically people that have specifically been approved by the user to receive alerts and digests. Followers may include family, close friends, medical providers, care givers, care organizations, and/or other members of the user's support network. In some embodiments, Activity Monitoring System 100 includes logic and a user interface (not shown) configured for a user to approve followers and to indicate what information each follower may receive.

Alert Logic 755 is optionally configured to open a communication channel between the user and a follower. For example, Alert Logic 755 may be configured to automatically place a call between Monitored Device 110A and a follower. This communication channel can include audio or video communication between the user and follower.

Activity Monitoring System 100 optionally further includes Alert Cancellation Logic 760 configured for the user to cancel an alert before it is sent to followers of the user. Alert Cancellation Logic 760 is optionally part of Confirmation Logic 147. For example, Alert Cancellation Logic 760 may be configured to display a message on Monitored Device 110A stating that an alert will be sent to all followers because Monitored Device 110A has been detected as being at a hospital or may have been in a car crash. This message may give the user a specific time in which to cancel the alert or to select which followers receive the alert. Alert Cancellation Logic 760 may also be configured for a user to include an audio or text message in an alert. For example, the user may wish to send a message stating, “I'm just at the police station for a fundraising event.”

In some embodiments, cancellation of an alert is considered a false positive event and is used by Threshold Logic 750 to adjust one or more threshold levels. For example, if a new user cancels a lot of alerts, then thresholds may be expanded to reduce the false positive rate. If an alert is cancelled, then the activity that resulted in the alert may be used to adjust the expected activity of the user. In one example, a user may volunteer at a hospital every Saturday morning, then a hospital visit at this time may become part of their expected activity. After a few cancellations on Saturday morning of alerts resulting from hospital visits, the cancellations are used to improve the prediction of the future activity levels of the user. In some embodiments, Question Logic 195 is configured to select questions in order to determine why an alert was cancelled.

Activity Monitoring System 100 optionally further includes Device Registration Logic 765. Device Registration Logic 765 is configured to register Monitored Devices 110 and optionally to associate these devices with specific users. For example, a user may use Device Registration Logic 765 to identify their home security system, their smartphone, their tablet computer, and their vehicle global positioning system (e.g., OnStar, GPS navigation, or airbag system) as including devices that represent their activity levels. As discussed elsewhere herein, some Monitored Devices 110 may represent the activity of more than one user. Thus, a TV remote, toilet use sensor or home security sensor may be associated with several people living in the same residence. In some embodiments, the location of one registered device can affect how use of a second device is interpreted by Activity Analysis Logic 130. For example, a TV remote may be registered as representing activity of both a husband and wife. However, if a smartphone associated with a wife has traveled to a distant city, then use (or lack thereof) of a TV remote may be treated as representing activity of just the husband.

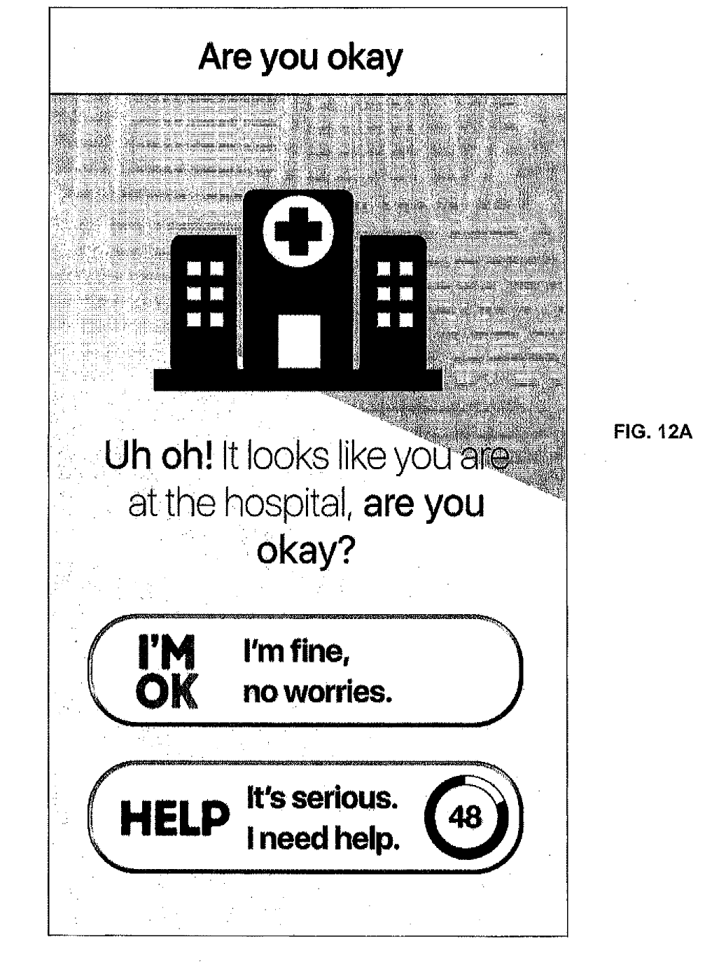

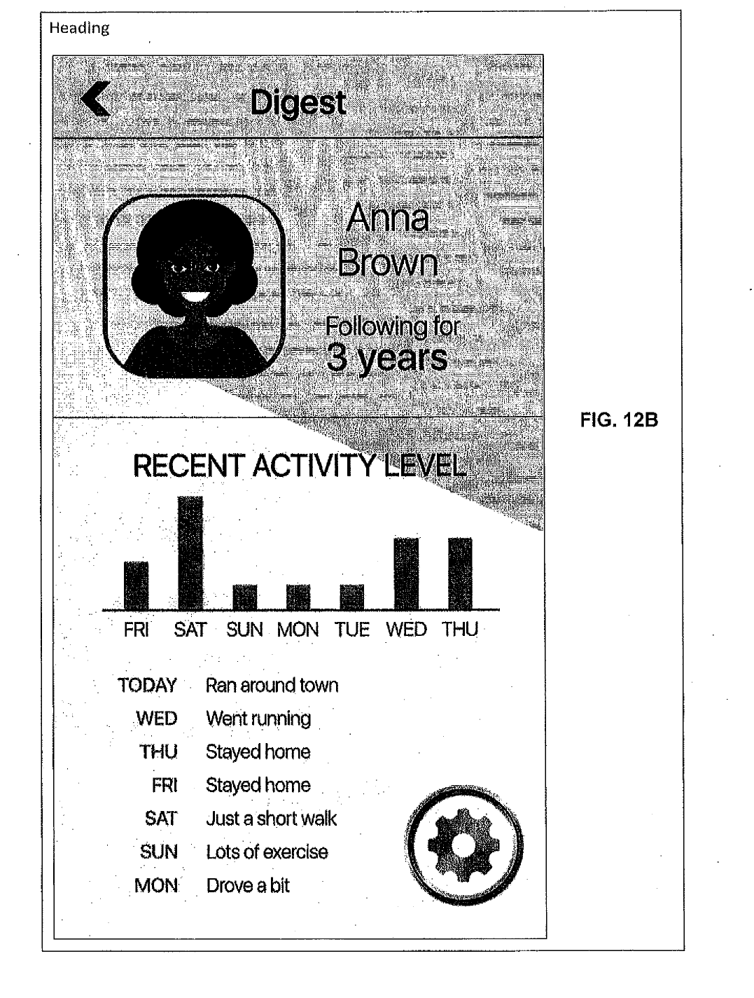

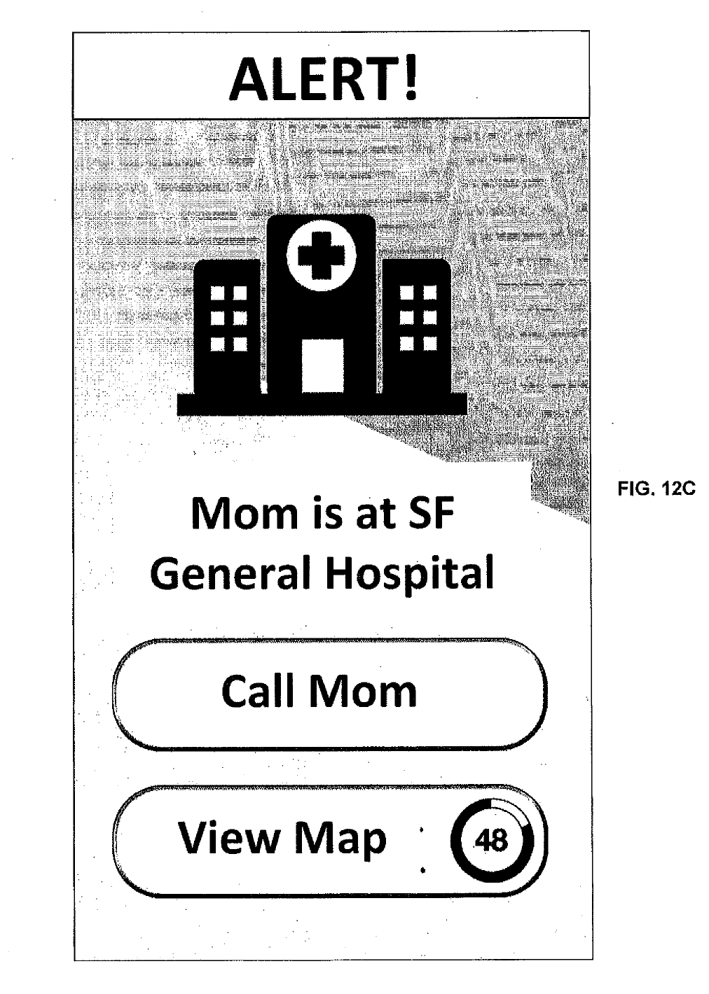

Activity Monitoring System 100 optionally further includes Digest Logic 770. Digest Logic 770 is configured do generate a digest that includes a summary of the activity of a user. The summary optionally includes filtered information about the activity level of the user. For example, it may include that the user spent time outside or was active without specifying exactly where the user went. In some embodiments, the daily digest includes a quantitative representation of the activity level of the user, such as a score, number or bar graph. The digest may be sent daily, weekly or on some other periodic basis. FIG. 12B illustrates an example of a daily digest. Users are optionally encouraged to improve their activity score.

FIG. 8 illustrates a device selection Interface 200, according to various embodiments of the invention. Such an Interface 200 may be generated by Device Registration Logic 765 and provided to a device of a user. Interface 200 is configured for the user to register devices that generate data that may be indicative of their activity. For example, Sasha's iPhone, and the other devices listed, may each be embodiments of Monitored Devices 110. Some of the devices illustrated in FIG. 8 are registered to more than one user. The registration process performed by Device Registration Logic 765 may include providing IP addresses, device serial number, phone numbers, and or the like. In some embodiments, registration includes connecting each of the devices to a central server that includes Device Registration Logic 765. In some cases, registration includes connecting a third-party service, such as a home security monitoring services, to Activity Monitoring System 100.

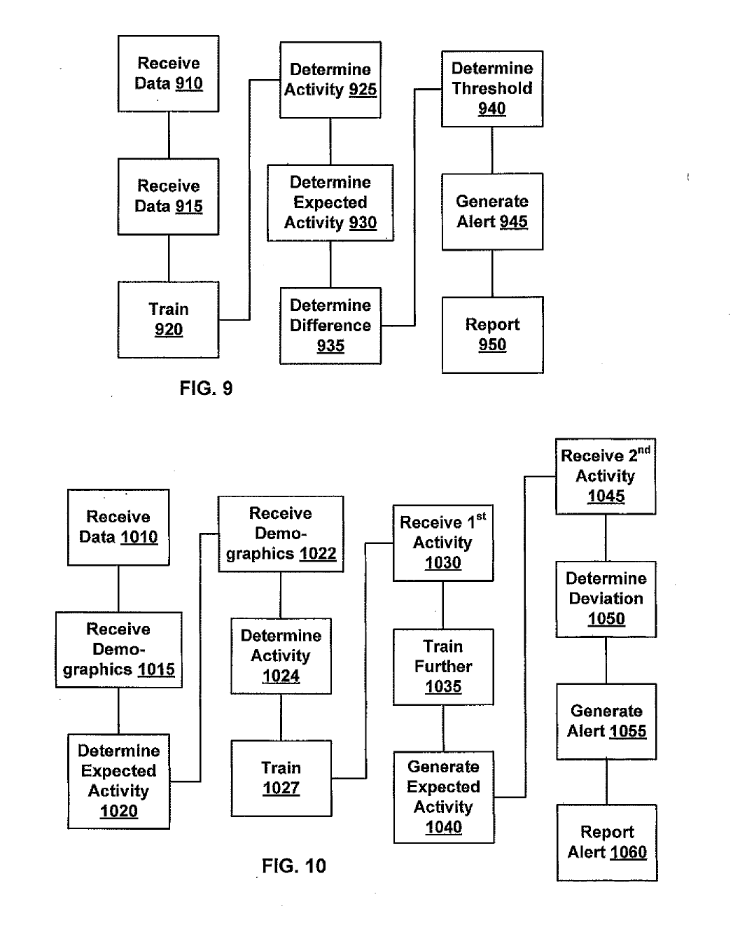

FIG. 9 illustrates methods of generating an alert, according to various embodiments of the invention. In these methods, a sensor data is received from multiple devices and used to determine an activity level of a user. The determined activity level is compared with an expected activity for the user, and if the comparison shows a difference greater than a threshold, then an alert may be generated and sent to followers. The received sensor data is optionally further used to train a machine learning to predict the expected activity for the user and/or to train a machine learning system to interpret what activity is represented by specific data received from one or more sensors.

In a Receive Data Step 910 data is received from Monitored Device 110A. This data is generated from signals produced by Sensor 715A and/or additional sensors included in Monitored Device 110A. The data is optionally received via Network 115.

In an optional Receive Data Step 915 data is received from Monitored Device 110B. This data is generated from signals produced by a Sensor 715B (not shown) and/or additional sensors included in Monitored Device 110A.

Note that additional receive data steps like Steps 910 and 915 may be included in the methods illustrated by FIG. 9. These receive data steps may include receiving data from additional devices such as Monitored Device 110C, etc. The data received in Steps 910 can be from different types of Sensors 715 and/or different types of Monitored Devices 110. Steps 910 and 915 may occur contemporaneously or may occur at different times. In some embodiments, Steps 910 and/or 915 occur at regular time intervals, such as once per minute, hour, day or week. In some embodiments, Steps 910 and/or 915 are triggered by specific events, such as detection of unusual acceleration of a smartphone (such as would indicate an accident), lack of use of Monitored Device 110A for a time period, presence of Monitored Device 110A at an unexpected location (e.g., hospital or police station), and/or any of the other acute events discussed herein.

In an optional Train Step 920 the data received in Steps 910, 915 and/or additional receive data steps is used to train a machine learning system, such as Machine Learning System 735 and/or Machine Learning System 745. The training may be directed toward determining what activities may be represented by the sensor data received in Steps 910 and 915; and/or the training may be directed toward determining activities for a specific user and/or a cohort of users. As noted elsewhere herein, the training may also use answers to questions provided to caregivers, the answers being associated with the activities and/or sensor data.

In a Determine Activity Step 925, the data received in Steps 910, 915 and/or additional receive data steps are used to determine actual activity of the user. Determine Activity Step 925 is optionally performed using Machine Learning System 735. In some embodiments, all or part of Determine Activity Step 925 is performed on Monitored Device 110A. For example, initial processing of sensor data may occur on Monitored Device 110A and further processing of the sensor data may occur on a server included in Activity Monitoring System 100. This server may include a Microprocessor 180 configured to execute part of Activity Analysis Logic 730. In some embodiments, communication of the data from Monitored Device 110A in Receive Data Step 910 is dependent on a result of the initial processing of sensor data that occurs on Monitored Device 110A. For example, Sensor 715A may be configured to detect acceleration, and only when the detected acceleration is indicative of a reportable event are the data communicated from Monitored Device 110A in Receive Data Step 910. In various embodiments, reportable events can include any one or more of the acute events discussed herein.

In a Determine Expected Activity Step 930, the expected activity of the user is determined. As noted elsewhere herein, the expected activity may be determined using Machine Learning System 745. Further, the expected activity may be determined using actual activity of the user and/or activity of a cohort of users of which the user is a member. The expected activity may be determined prior to any of the other steps illustrated in FIG. 9. In some instances, expected activity is based on presumptions about normal activity. For example, most people don't decelerate from 45 to 0 MPH in less than 3 seconds. The expected activity can include many different dimensions, as discussed herein, such as location, physiological functions, travel, exercise, steps taken, movement patterns, sleep patterns, etc. Expected activity may include temporal dependencies. For example, the activity expected on Sunday morning may be different than that expected on Saturday night. Or, staying in bed for 9 hours may be more expected at night than during the day.

In a Determine Difference Step 935, one or more differences between the expected activity of the user (from Step 930) and the actual activity of the user (from Step 925) is determined. Determine Difference Step 935 is optionally an embodiment of Compare Step 330 or Determine Deviation Step 1050. The differences between the expected and actual activity is optionally represented by a cosine distance is a multi-variable space. Determine Difference Step 935 can include differences in a single dimension, and in addition differences between expected correlation between different dimensions. For example, if a user either plays golf or paintball every Sunday then an activity level that includes neither golf nor paintball may be unexpected. A lack of movement detected by a home security system may be more likely when the user's smartphone is visiting at a friend's residence, relative to when the smartphone is home.

In a Determine Threshold Step 940, a threshold is determined for the difference between expected activity of the use and the actual activity as measured using Sensors 715. This threshold can include multiple dimensions and/or can be determined for individual dimensions or for dimensions in combination. For example, a threshold may be determined for a combination of, or function of, steps taken in a day and time spent on social networking websites. In some embodiments, the threshold is dynamic. For example, the threshold may be dependent on how accurately the estimated activity is (believed to be) known and/or how accurately the interpretation of the data from Sensors is believed to represent an actual activity of the user. Determine Threshold Step 940 is optionally performed using Threshold Logic 750. Determine Threshold Step 940 is optional if thresholds are already known or are set to default values.

In a Generate Alert Step 945 an alert is generated in response to one or more of the thresholds of Determine Threshold Step 940 being greater than the respective one or more differences determined in Determine Difference Step 935. The alert typically includes reasons that the alert was generated. For example, if the user's activity has declined over several days, the alert may state this fact. The alert may also suggest possible remedies or that the follower contact the user being followed. Contact and location information regarding the user may also be included in the alert.

Generate Alert Step 945 optionally includes providing the user with an opportunity to cancel the alert, using Alert Cancellation Logic 760. Cancellation of alerts can reduce incorrect alerts and gives the user a final step of control over their privacy. In some embodiments, the user may cancel delivery of the alert to some followers but not other followers. For example, the user may wish to notify only a subset of their followers that they are at a police station. The length time provided to cancel the alert is optionally dependent on the type or and/or reason for the alert. For example, a reduction in general physical activity may result in an alert that can be cancelled within an hour, while an alert resulting from a suspected car accident may only provide a 3-minute window to cancel. The contents and/or delivery of the alert are optionally responsive to a magnitude of the difference between the expected and actual activity, or a magnitude by which a threshold is acceded.

In a Report Step 950, the alert, if not cancelled, is sent to followers of the user. Depending on the type and reason for the alert, the alert may be sent to different sets of followers. For example, an alert that the user is at a hospital may be sent so a first set of followers and an alert that the user has been coughing may be sent to a different set of followers. The user may predetermine these sets of followers. The alert may be included as part of a daily digest or may be sent to a follower via any other communication means. For example, the alert may be sent as an instant message or e-mail to a follower's smart phone. The method of communication is optionally dependent on an urgency of the alert. Generate Alert Step 945 and Report Step 950 are optionally embodiments of Alert Step 340.

FIG. 10 illustrates methods of training a machine learning system for a user, according to various embodiments of the invention. These methods may be used to train either Machine Learning System 735 or Machine Learning System 745. In other words, the method may be used to train a machine learning system to generate an expected activity for a user or to train a machine learning system to estimate actual activity based on data from Sensors 715. In either case, an initial state for the machine learning system is selected/generated based on information that is not specific to just one user. Following this initial state, data gathered from Monitored Devices 110 of the user is used to further train the machine learning system. The steps discussed below are related to training a system to determine expected activity levels, however, the may be adapted to training for sensor data interpretation.

In a Receive Data Step 1010, activity data is received regarding multiple users. The data is optionally based on sensor data received from multiple instances of Monitored Devices 110 associated with the multiple users. The data may be collected over an extended period. The received activity data is optionally annotated by answers to questions selected using Question Logic 195.

In a Receive Demographics Step 1015, demographics are received regarding the multiple users. The demographics may include gender, age, weight, residence location, profession, medical history, and/or any other data that may be used to divide the users into different cohorts. The demographics and activity data are associated with specific users.

In a Determine Expected Activity Step 1020, an expected activity levels for the multiple users are determined. The expected activity levels are optionally dependent on the demographics of the multiple users and optionally include one or more statistical distributions of activity levels as a function of the demographics. The expected activity may be determined by a statistical analysis of activity data received in Receive Data Step 1010. The expected activity may be represented by a state of a machine learning system, e.g., Machine Learning System 745 trained using the activity data.

In a Receive Demographics Step 1022, demographics of a first user are received. The first user is not necessarily a member of the multiple users. The demographics may be received by having the first user register for an account, from medical data, by having the first user enter data on a user interface, and/or the like.

In a Determine Activity Step 1024, an expected activity specific to the first user is determined. This determination may be made by selecting the expected activity, from among the expected activity levels for the multiple users, as determined in Determine Expected Activity Step 1020. The selection is based on the demographics of the first user. For example, an expected activity level across multiple dimensions of activity may be determined based on a statistical correlation between the demographics and the activity distributions of the multiple users. Alternatively, the expected activity may be determined by retrieving a state of Machine Learning System 745 associated with the demographics. In Determine Activity Step 1024 the expected activity is based on the demographics of the first user, and not necessarily on any sensor data received regarding the first user.

In an optional Train Step 1027, Machine Learning System 745 is trained to predict the expected activity of the first user. In some embodiments, the training is based on the activity of the multiple users, the demographics of the multiple users and the demographics of the first user. In some embodiments, the training produces a state of Machine Learning System 745 representative of the expected activity level of the first user. Train Step 1027 may be performed by a third party and received in Determine Activity Step 1024, in which case Train Step 1027 is optional.

In a Receive 1^{st} Activity Step 1030 first activity data regarding the first user is received, the activity data representing an activity level of one or more dimensions. This activity data may be determined based on sensor data produced by Sensor 715A. The sensor data is optionally interpreted using Machine Learning System 735. This activity data is optionally augmented by association with answers provided by caregivers, and the activity may be determined using Machine Learning System 735, Machine Learning System 745, and/or Rule Logic 747, as described elsewhere herein. In some embodiments, augmentation can be retroactively based on an actual medical diagnosis.

In a Train Further Step 1035, Machine Learning System 745 is further trained using the activity data received in Receive 1^{st} Activity Step 1030. Steps 1030 and 1035 may be repeated, 2, 3 or more times. As such, the training may employ temporal and/or time dependent machine learning techniques. These repetitions are indicated at Receive 2^{nd} Activity Step 1045, etc. in FIG. 10.

In a Generate Expected Activity Step 1040, a new expected activity of the first user is generated using Machine Learning System 745. This expected activity is based on both the state of Machine Learning System 745 generated/trained on the activity of the multiple users (Step 1027) and on the further training that occurs with each execution of Step 1035.

In a Receive 2^{nd} Activity Step 1045, further activity data indicating an activity level of the user is received. Receive 2^{nd} Activity Step 1045 is an embodiment of Receive 1^{st} Activity Step 1030 that occurs later in time.

In a Determine Deviation Step 1050, it is determined that activity data received in a Receive 2^{nd} Activity Step 1045 represents a deviation from the expected activity of the user.

In a Generate Alert Step 1055, an alert is generated in response to the deviation. Generate Alert Step 1055 is optionally an embodiment of Generate Alert Step 945. The alert is optionally dependent on embodiments of Determine Difference Stop 935 and/or Determine Threshold Step 940, as illustrated in FIG. 9.

In a Report Step 1060, the alert is reported to one or more followers of the user. Report Step 1060 is optionally an embodiment of Report Step 950 of FIG. 9.

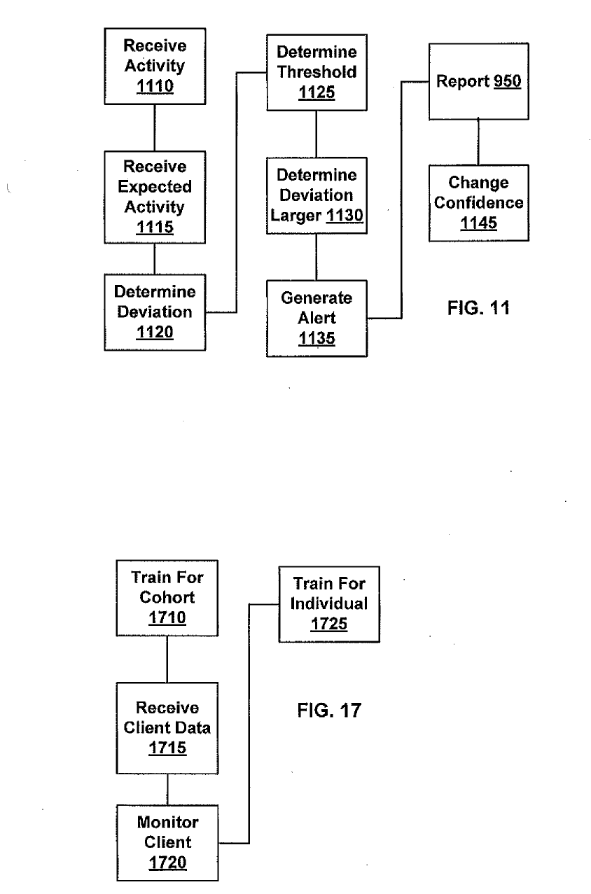

FIG. 11 illustrates methods of generating an alert based on a dynamic threshold. This dynamic threshold is optionally generated using Threshold Logic 750. The dynamic threshold can include multiple dimensions and may be different for each dimension. The dynamic threshold is used to determine if a deviation from expected activity is enough to generate an alert. Different dimensions of the dynamic threshold may change by different amounts and/or in different directions.

In some embodiments, a threshold can have multiple levels that result in different actions when exceeded. For example, an initial threshold level may result in the inclusion of concerning activity changes in a daily digest, while a higher threshold level may result in generation of a real-time alert. In an illustrative example, a blood glucose level of that drops to 70 mg/dL may result in a notation in a daily digest, while a blood glucose level that drops to 50 mg/dL may result in an urgent real-time alert.

In a Receive Activity Step 1110 an activity level of a user is received. As noted elsewhere herein, the activity level may be determined using Activity Analysis Logic 730 and data received from Sensors 715. Receive Activity Step 1110 optionally includes Steps 910, 915, 920, 925, described with respect to FIG. 9.

In a Receive Expected Activity Step 1115 an expected activity is received. Receive Expected Activity Step 1115 optionally includes Determine Expected Activity Step 930, discussed with respect to FIG. 9. The expected activity may be based on an output of Machine Learning System 745, activity levels of the user, and/or activity levels of multiple users. For example, if one of the methods of FIG. 10 is used to train Machine Learning System 745, then the expected activity may be initially based on the activities of a cohort of multiple users, and after further training of Machine Learning System 745 may be later and further based on activities of the user.

In a Determine Deviation Step 1120 it is determined that activity level of the user received in Receive Activity Step 1110 represents a deviation from the expected activity of the user as received in Receive Expected Activity Step 1115. Determine Deviation Step 1120 optionally includes an embodiment of Determine Difference Step 935, discussed with respect to FIG. 9. The deviation may be of different amounts for different dimensions of the activity.

In a Determine Threshold Step 1125 a threshold for the deviation is determined. The threshold typically different for different dimensions of activity and is dynamic. A dynamic threshold is one that may vary depending on different criteria. In various embodiments, the threshold varies as a function of time, as a function of an expected confidence (accuracy) of the expected activity level, as a function of an expected accuracy of the received activity level, as a function of an amount of activity data received for the user, as a function of the demographics of the user, as a function of the medical history of the user, and/or the like. Determination of a threshold is optionally responsive to answers to questions selected using Question Logic 195. For example, an answer to a question may explain a deviation or indicate that a deviation is likely to indicate a health problem. In an illustrative example, if a monitored user is detected getting up several times at night, then a question about how well the user slept may be selected. An answer to that question of “there was a party next door” may be indicative that getting up is not the result of an undesirable health state, while an answer “I keep feeling like I have to pee but cannot” may be indicative of the likelihood of a health problem that warrants an alert be sent. Thresholds determined in Determine Threshold Step 1125 may, therefore, be based on responses to selected questions.

The expected confidence of the expected activity level may be a multi-dimensional statistical function (e.g., probability distributions), and may be based on an amount of training received by Machine Learning System 745, on a meta-analysis of Machine Learning System 745, on historical accuracy of Machine Learning System 745, on demographics of the user, how much of the training data is specific to the user, on the accuracy of past expected activity levels, and/or the like. For example, in some embodiments, it may be determined statistically that after an amount of training X that dimension D of expected activity has a Y % probability of fitting within a specific distribution. Such statistical determination may be based on the training of many instances of Machine Learning System 745 for many individual users.

The expected accuracy of the received activity level may be dependent on the quality and/or quantity of sensor data received, the age of the sensor data, the type of sensor data received, the training of Machine Learning System 735 (possibly dependent on the same factors relevant to training of Machine Learning System 745 discussed above), and/or the like.

Medical history may be used to dynamically vary the dynamic threshold for one or more dimensions. For example, if a user is diagnosed with Type I diabetes, their thresholds for blood glucose levels may be adjusted for this medical condition.

In a Determine Deviation Larger Step 1130, it is determined that the deviation of Determine Deviation Step 1120 is larger than the dynamic threshold in one or more dimension of activity. In some embodiments, correlations between multiple activity dimensions are considered in this determination.

In a Generate Alert Step 1135 an alert is generated in response to the determination of Step 1130. Generate Alert Step 1135 is optionally an embodiment of Generate Alert Step 945 or Generate Alert Step 1055. The Alert may then be reported in instances of Report Step 950 or Report Step 1060.

In an optional Change Confidence Step 1145 a confidence in the accuracy of either the expected accuracy of the received activity level and/or the expected confidence of the expected activity level may be changed. This change in confidence is optionally used to change associated dimensions of the dynamic threshold. The confidence may be Changed, for example, if the alert is canceled by the user, if the user provides feedback on the alert, and/or the like. For example, if and alert resulting from the same cause is cancelled by a user several times, then the threshold for the activity dimension that caused the alert may be increased. As such, thresholds may be dynamically responsive to alert cancellations made using Alert Cancellation Logic 760.

The methods illustrated in FIGS. 9-11 are optionally performed in combination with those performed in FIGS. 3-6.

FIGS. 12A, 12B and 12C illustrate a real-time alert, an alert cancellation interface and a digest, according to various embodiments of the invention. In FIG. 12A an alert cancellation interface is shown. This interface would be presented to a user before an alert is sent to followers. The interface provides a button configured to cancel the alert by saying “I'm fine,” and a button configured to say “Help” and send the alert immediately (rather than wait for a delay). The interface shown in FIG. 12A may be displayed on Monitored Device 110A or Peripheral 172.

In FIG. 12B a digest of a user's activity is displayed. This digest shows recent activity levels in the form of a daily bar chart and provides a brief text summary of daily events. This digest is the type that may be received by a follower on Monitoring Device 120A. The digest is configured to give high level activity information while maintaining privacy of the monitored user.

In FIG. 12C an alert is displayed. This alert indicates a specific event that “Mom is as SF General Hospital.” Such an alert may be received by a follower on Monitoring Device 120A.

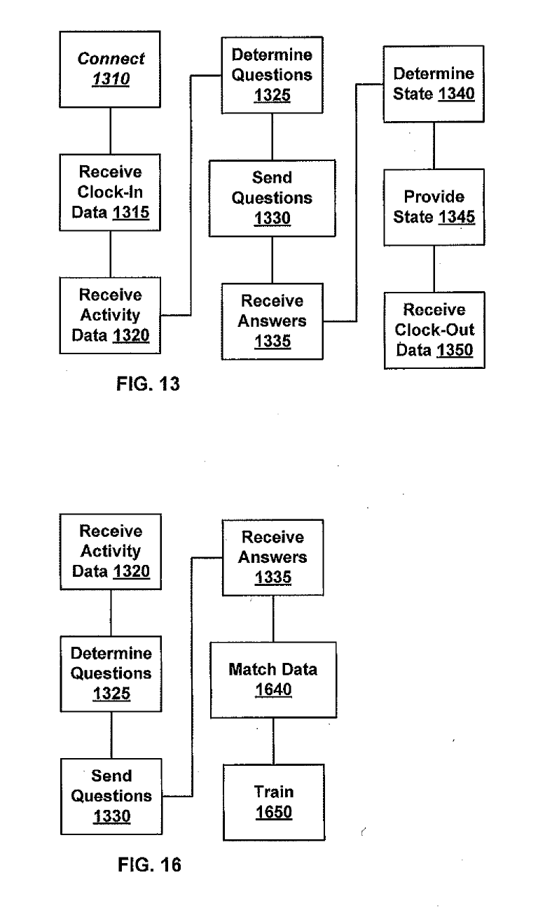

FIG. 13 illustrates a method of determining a state of health, according to various embodiments of the invention. In this method, Question Logic 195 is used to select questions to be presented to a cared for user. These questions are selected to assist in the determination of the health state. In response to the questions, answers are received. These answers may be then used as one of the inputs to determining the health state and/or training of the various machine learning systems discussed herein. The steps illustrated in FIG. 13 are optionally performed in alternative orders.

In a Connect Step 1310, a connection is made to a remote device associated with one or more cared for users. These users are optionally clients of a home care, nursing home, hospital and/or other care organization. For example, the remote device may be an instance of Monitored Device 110A associated with a senior or disabled person. The connection is optionally made through Network 115.

In an optional Receive Clock-In Data Step 1315 data regarding check-in of a caregiver is received. This data typically represents the start of a shift for the caregiver during which services are provided to the client. The check-in data can include an identity of the caregiver, an identifier of the client or device associated with the client, and a time of day. The check-in of the caregiver may be used to alter the processing of data received from Monitored Device 110A. For example, the processing may now account for the fact that sensors will detect activity of the caregiver as well as the client. Further, the processing may now be used to confirm that the caregiver is present and performs certain tasks.

In some embodiments, Monitoring Device I20 includes an interface configured for other parties to declare their presence. For example, a visitor to a senior's home may check in and out to declare their presence, and the analysis of sensor data and activities (e.g., by Activity Logic 135 or Activity Analysis Logic 730) may be adjusted for the presence of additional persons in the home.

In a Receive Activity Data Step 1320, activity data is received. The activity data may be generated using Machine Learning System 735 based on sensor data received from Monitored Device 110A and/or Peripheral 172. In an illustrative example, sensor data is received by Machine Learning System 735 on Management Server 123 or Monitored Device 110A. Machine Learning System 735 processes the received sensor data and outputs one or more activities of a monitored user. This output of one or more activities is provided to Machine Learning System 745 or Rule Logic 747 in Receive Activity Data Step 1320. The received activity may represent movement of the monitored user, detected by a wearable and/or non-wearable device. The received activity may be associated with one person in a multi-person household.

In a Determine Questions Step 1325, Question Logic 195 is used to determine one or more questions to be provided to a caregiver, optionally via Network 115 and Caregiver Interface 158. This determination can include generating questions in real time and/or selecting questions from an existing set of questions. As described elsewhere herein, the selection of questions can be based on any of a wide variety of criteria and can be performed to reach one or more different goals. These goals can include, for example, receiving answers of greatest value in identifying a health state of a cared for user, receiving answers of greatest value in training any of the machine learning systems discussed herein, auditing performance of a caregiver, and/or the like. A question may be selected to generate an answer with (balanced) value in both identifying a health state for the user and further training Machine Learning Systems 735 or 745.

The questions selected in Determine Questions Step 1325 are optionally selected to confirm a health state determined using Machine Learning System 745 and/or Rule Logic 747. The answers to these questions can be associated with the health statement and further used to train Machine Learning System 745 and/or Question Logic 195.

The number of questions selected is optionally based on a question budget. This budget may change depending on answers received. For example, if an answer increases the value of another follow-up question, then the total number of questions selected may be increased. In a specific case, a question budget for a user is four questions per day. If the answers to these questions indicate possibility of an undesirable health state, then the budget may be increased to select questions having value in determining if that possibility is relatively high or low. Specifically, if initial answers indicate possibility of pneumonia, then additional questions may be selected to confirm or eliminate this possibility. Note that, in typical embodiments, Monitoring System 100 and Activity Monitoring System 700 are configured to determine if a health state should be investigated by a medically qualified professional, not to make an actual diagnosis.

In a Send Questions Step 1330, the one or more questions selected in Determine Questions Step 1320 are sent to Monitored Device 110A for display on Caregiver Interface 158, or some other remote device. The questions can be sent as a group or one at a time. Send Questions Step 1330 is optionally initiated by the receipt of clock-in data in Receive Clock-in Data Step 1315. This allows the caregiver to ask the questions at the start of a shift or during certain times.

In a Receive Answers Step 1335, one or more answers to the question(s) sent in Send Questions Step 1330 are received. The answers can include typed text, recorded audio, text generated from a voice to text system, identities of checked boxes, output of a medical device, images, and/or any other answer types discussed herein.

Determine Questions Step 1325, Send Questions Step 1330 and Receive Answers Step 1335 may be repeated multiple times and in different orders. For example, in some cases several questions are selected at once and sent before answers are received. Alternatively, in some cases a second question is not selected and/or sent until after an answer to a first question is received. In this case, the answer to the first question can be considered in the selection of the second question.

In a Determine State Step 1340, one or more health states of a monitored user are determined based on the activities received in Receive Activity Data Step 1320 and/or the answers received in Receive Answers Step 1335. The one or more health states may be determined using, for example, Machine Learning System 745 and/or Rule Logic 747. As discussed elsewhere herein, the determination of health states may be based on a wide variety of information including, for example, sensor data, determined activities, expected activities, medical history, answers to questions sent to Monitored Device 110A, and/or the like.

In an optional Provide State Step 1345, the health state(s) determined in Determine State Step 1340 are provided to the cared for user, caregivers, caregiver managers (e.g., a caregiver agency, nursing home, hospital, etc.), followers of the user, family members, and/or the like. Provide State Step 1345 optionally includes determining whether to provide the health state to various parties dependent on the identity, severity, and/or importance of the health state. For example, a health state of a possible skin cancer may be sent only to care organization and qualified medical personnel; while a fall may be reported to the care organization and family members.

In an optional Receive Clock-Out Data Step 1350, data regarding check-out of a caregiver is received. This data typically represents the end of a shift for the caregiver, during which services were provided to the client. The check-out data can include an identity of the caregiver and a time of day. The check-out of the caregiver may be used to alter the processing of data received from Monitored Device 110A. For example, the processing may now account for the fact that sensors will not detect activity of the caregiver, after a short delay.

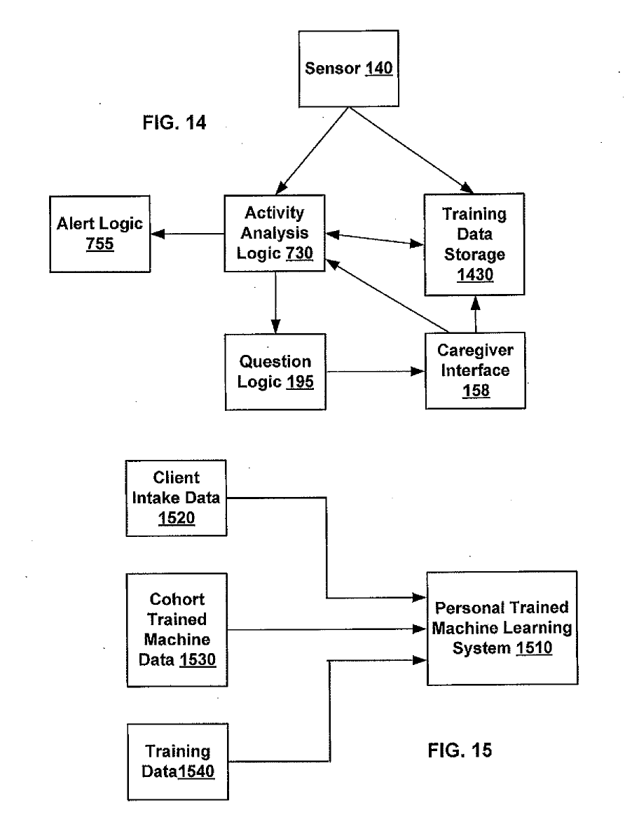

FIG. 14 illustrates data flow between various elements of an activity monitoring system, such as Activity Monitoring System 700, according to various embodiments of the invention. As illustrated, sensor data generated at Sensor 140, Peripheral 172, or Sensors 715 are sent to Activity Analysis Logic 730 and a Training Data Storage 1430. At Activity Analysis Logic 730 the sensor data is used to determine an activity and/or health state of a cared for user. The determined activity is sent to Question Logic 195 for selection of one or more questions based on the activity of the user. The determined activity may also be sent to Training Data Storage 1430. Questions selected based on the determined activity (and/or the sensor data) are sent to Caregiver Interface 158. Answers to the questions are sent from the Caregiver Interface 158 are sent to Training Data Storage 1430. At Training Data Storage 1430 the answers are associated with the determined activity and/or the received sensor data. The associated answers and determined activity and/or sensor data are sent to Activity Analysis Logic 730 for further training of machine learning systems therein (e.g., Machine Learning Systems 735 and/or 745). The training is optionally performed by training logic disposed within Machine Learning Systems 735 and/or 745. Various steps in this process may be repeated in a positive feedback look, in which activities and/or sensor data augmented by caregiver input is used to improve the performance of the machine learning systems to determine activities and/or health states. Finally, Analysis Monitoring System 730 is used to determine a health state from the determined activity(ies). This health state can be sent to Alert Logic 755 for the purposes of sending an alert based on the health state. The health state can also be confirmed by selection of further questions. This confirmation can also be used for training of Machine Learning System 745 and/or Question Logic 195, via the same positive feedback look discussed elsewhere herein.

FIG. 15 illustrates data flow in personalized training of a Personal Trained Machine Learning System 1510, according to various embodiments of the invention. A personal trained machine learning system is one trained for a specific individual. Personal Trained Machine Learning System 1510 can include Machine Learning System 735 and/or Machine Learning System 745. The inputs to Personal Trained Machine Learning System 1510 can include at least two of Client Intake Data 1520, Cohort Trained Machine Data 1530, and Training Data 1540. Client Intake Data 1520 can include, for example, data collected by a care organization when initiating care for a user (client), medical history, a care history, genetic data, epigenetic data, environmental data, a care plan, a recovery plan, a post-operative plan, demographic information of the user, and/or the like.

Cohort Trained Machine Data 1530 includes, for example, data characterizing a machine learning system trained using training data representative of a cohort of users. The data characterizing a machine learning system can include structure, weightings and connections between nodes of a neural network. The cohort of users may be defined by demographics and/or medical history. For example, a cohort may include non-smoking women between ages 70 and 74, who have had breast cancer.

Training Data 1540 typically includes sensor data, determined activities, and/or health states, as annotated by caregiver provided answers. As discussed above, such data may be stored in Training Data Storage 1430. The data may be annotated using the methods illustrated by FIG. 13. For examples, the annotations can include answers to questions selected based on sensor data or determined activities.

Personal Trained Machine Learning System 1510 may be trained for a specific user by additional training of specific layers within a neural network previously trained using cohort data. These specific layers are optionally disposed at the input or output of the neural network. Some embodiments include a hierarchy of layers to be trained using different data.

FIG. 16 illustrates methods of training a machine learning system, according to various embodiments of the invention. These methods may be used to train Machine Learning Systems 735 and/or 745. Receive Activity Data Step 1320, Determine Questions Step 1325, Send Questions Step 1330, Receive Answers Step 1335 are discussed elsewhere herein, for example with respect to FIG. 13. In a Match Data Step 1640, the answers received in Receive Answers Step 1335 are matched to the activities received in Receive Activity Data Step 1320 and/or to health states determined therefrom. The matched data represents caregiver annotated activity data and/or sensor data. This matched data is optionally stored in Training Data Storage 1430.

In a Train Step 1650, the matched data is used to train a machine learning system, such as Machine Learning Systems 735 and/or 745. In some embodiments, the machine learning system that is trained in Train Step 1650 is the machine learning system used to generate the activity data received in Receive Activity Data Step 1320. In some embodiments, the machine learning system that is trained in Train Step 1650 is also, or alternatively, the machine learning system that is used to determine a medical state from the received activity data. Training is optionally performed using training logic included in each of the machine learning systems. The trained machine learning system(s) are optionally used to determine further activities and/or health states, questions can be selected based on these further activities and/or health states, and the process is repeated in a positive feedback loop.

FIG. 17 illustrates methods of personalized training of a machine learning system, according to various embodiments of the invention. These methods are optionally used in Train Step 1650. The machine learning system trained can include Machine Learning Systems 735 and/or 745. The methods illustrated in FIG. 17 are optionally embodiments of the methods illustrated in FIG. 10.

In a Train for Cohort Step 1710, a machine learning system is trained for a cohort of clients. The cohort may be characterized by a demographic such as age, gender, diagnosed disease, health history, and/or the like. The training can be performed using annotated sensor data and/or annotated health state data, as described elsewhere herein. In a Receive Client Data Step 1715, data is received regarding an individual user. The received data identifying the user as a member of the cohort.

In a Monitor Client Step 1720, the user is monitored using Monitored Device 110A and/or Peripheral 172. This monitoring includes the receipt of sensor data and the determination of one or more activity based on the sensor data. Monitor Client Step 1720 optionally further includes determination of a health state of the user based on the sensor data and/or determined activity(ies). The determination of the activity and/or health state is optionally confirmed and/or annotated using questions selected for presentation to a caregiver.

In a Train for Individual Step 1725, the machine learning system, trained for the cohort, is further trained based on the sensor data, determined activity data, and/or determined health state. A goal of this training is to make the machine learning system more accurate with regards to identifying activities and/or health states for the user. Train for Individual Step 1725 may include training an input or output filter to a neural network and/or training a segment of a neural network.

As a result of Train for Individual Step 1725, the machine learning system, e.g., Machine Learning Systems 735 and/or 745, are trained based on both activity of the cohort of clients and the detected activity of the individual client/user. The machine learning system can be further trained to identify if an activity, trained to determine a health state of the user, and/or trained to generate an alert if a subsequent activity of the individual user deviates from an expected activity of the individual user.

Train for Individual Step 1725 is optionally followed by a repeat of Monitor Client Step 1720 in which the user is monitored to detect the subsequent activity, further activity, and/or subsequent health states.

Several embodiments are specifically illustrated and/or described herein. However, it will be appreciated that modifications and variations are covered by the above teachings and within the scope of the appended claims without departing from the spirit and intended scope thereof. For example, a Security System may be configured to change between modes; intruder detection mode and a health monitoring mode. Sending an alert due to inactivity is contrary to the operation of typical security systems. Machine Learning Systems 735 and 745 can include a deep learning system, a neural network, an adaptive expert system, and/or the like. The examples provided may be adapted to a peer-to-peer network that does not include a separate Management Server 123. The systems and methods disclosed herein may be adapted to the monitoring of pets or livestock. In further examples, the systems and methods discussed herein may be used to monitor medical treatments other than administration of pharmaceuticals or trauma recovery, e.g., physical therapy, exercise, consumption of a nutritional supplement, taking of pharmaceuticals, and/or dietary changes. Some embodiments are used to monitor a patient's compliance with a treatment and/or therapy. For example, a patent with bipolar disorder may be monitored to detect activity that would indicate that the patent was not taking prescribed medications.

Examples of IoT devices that may include Monitored Devices 110 include: Passive or active IR detectors, Wheelchair—travel measurement, Walking cane—steps, pressure measurement, Electronic personal attendant—robot—Amazon Alexia®, Bixby®, TV remote, baby monitor, Smart home devices, thermostat, oven fridge, physical therapy devices (e.g., weight machines or limb position measurement devices) microwave, coffee maker, car, entry locks, garage door, key Fob, finger ring, bracelet, Animal movement (collar)—dog has not gotten up all day, Wearable device, Medical device (pacemaker, insulin pump, pill container, Security system, Animal tracking (combined with electronic fence, livestock). Some instances of Monitored Devices 110 may be configured for delayed data transmission, when the device is plugged in or within radio range. Normally security systems provide notice when an event occurs, or perhaps a status ping to show that they are operational.

Some embodiments include a security system that also sends a notice when there is a lack of action or an unexpectedly low amount of action. In some embodiments, the security system is configured to report signals detected at specific sensors to a remote processing system. Some embodiments provide both home security and a health monitoring service. Sensors used to detect the presence of an intruder are also used to monitor activity of a user.

An alert system configured to detect when current activity of a user deviates from an expected activity, and to provide an alert to a remote client when the deviation is detected. The expected activity is based at least in part on a measured activity. The measured activity is optionally measured over time using at least one sensor. The alerts are sent when the deviation is greater than a dynamic threshold. The threshold represents a permitted difference between current activity of the user and the expected activity and is dynamic because it is dependent on a confidence to which the expected use and/or actual use are known. For example, in some embodiments, as the measured activity is measured over time a probability that the measured use represents a true expected use increase. In response to the increased probability the threshold is reduced.

This approach to alert generation results in a high initial threshold that is reduced over time to a lower value as a user's activity is monitored. The high initial threshold prevents alerts from being sent when they shouldn't be (false positive errors), relative to a system that initially used the lower threshold value. The change in threshold as a function of the probability increase can be based on a linear relationship, or on a stochastic model that attempts to achieve at least a desired false positive rate. The stochastic model may be based on measured activity of many users over time.

Further examples:

In an illustrative example, a new user is provided with one or more sensors configured to measure activity of the new user. Initially the expected activity is optionally based on a classification of the user. For example, the user may be assigned to a cohort based on their gender, age, wealth, weight, height, education, employment, and/or other demographic. The initial expected activity for the new user may then be assumed to be that of historical expected activity for members of the cohort. The historical activity for the cohort may be associated with an expected deviation (e.g., a standard deviation over a Gaussian distribution.) This deviation allows for a calculation that the expected activity of the cohort will represent the true expected use for the new user. It also allows for calculation of a threshold that will likely result in a false positive rate of less than a predetermined rate. For example, the predetermined rate may be set to one false positive (incorrect alert) per month.

As actual activity of the new user is measured over time. The measured activity can be used to modify their expected use, thus bringing the expected use closer to the true expected use. This also increases the probability that the estimated expected user activity level represents the true expected user activity level. The threshold can be reduced in response to this increased probability. Optionally, the threshold is reduced to keep or improve false positive rate.

The activity of a user can be measured in many different aspects. For example, assuming the sensor used to measure activity is an accelerometer in an Apple Watch®, the activity can be represented by the user's heart rate, several steps the user takes each day, and the frequency of sudden accelerations measured by the sensor. Dynamic thresholds are optionally set for each of these measurement types. The different types of activity are referred to herein as “dimensions” of activity.

Take for example, a new user that is a 30 years old overweight male. His initial expected activity may include taking 4000 steps per day and rarely experiencing rapid acceleration of the watch. There is a calculated probability that the initial expected activity represents his true activity. This probability is relatively low, resulting in a high threshold. On day 1 several potential alerts are detected but cancelled by the user. As a result, the thresholds are automatically raised. This reduces the number of false alerts. After measuring the new user's actual activity for a week, it is found that he walks 8000 steps a day, keeps his heart rate above 100 bpm for at least an hour a day and has a resting heart rate of 80, and experiences sudden accelerations over 50 times per day. This activity is summarized in a daily digest. The expected activity for this user has now been adjusted based on actual measured activity for this user. As confidence in the expected activity increases the thresholds can be lowered. If this user now has a day of only 2000 steps, this may be enough to cause an alert, or to at least be noted in a digest.

Some embodiments include small inexpensive sensor devices equipped with motion sensors and a radio frequency transmitter. The sensor devices are configured to send a radio signal responsive to movement or lack of movement. The sensor devices are optionally configured to be attached to common objects such as a laptop computer, bathroom door, bed, refrigerator door, dishwasher, TV remote, etc. to detect use of these devices. The sensor devices are optionally configured to be plugged into an AC power outlet.

Some of the sensor devices may be configured to report a lack of use during a predetermined period, while some of the sensor devices may be configured to report use. In either case, the reporting is made by radio frequency signal to a monitored device. The monitored device is configured to determine if activity has not been detected by a set of sensor devices during a period and if a lack of detection occurs in all of the members of the set, to then send an alert to a remote destination. The alert indicating the lack of activity in all members of the set.

For an individual sensor device to report a lack of use typically includes: a clock, logic to measure predetermined period and determine that it has been exceeded, and a transmitter to report the lack of use. This sensor device may also include a circuit configured to receive setup data.

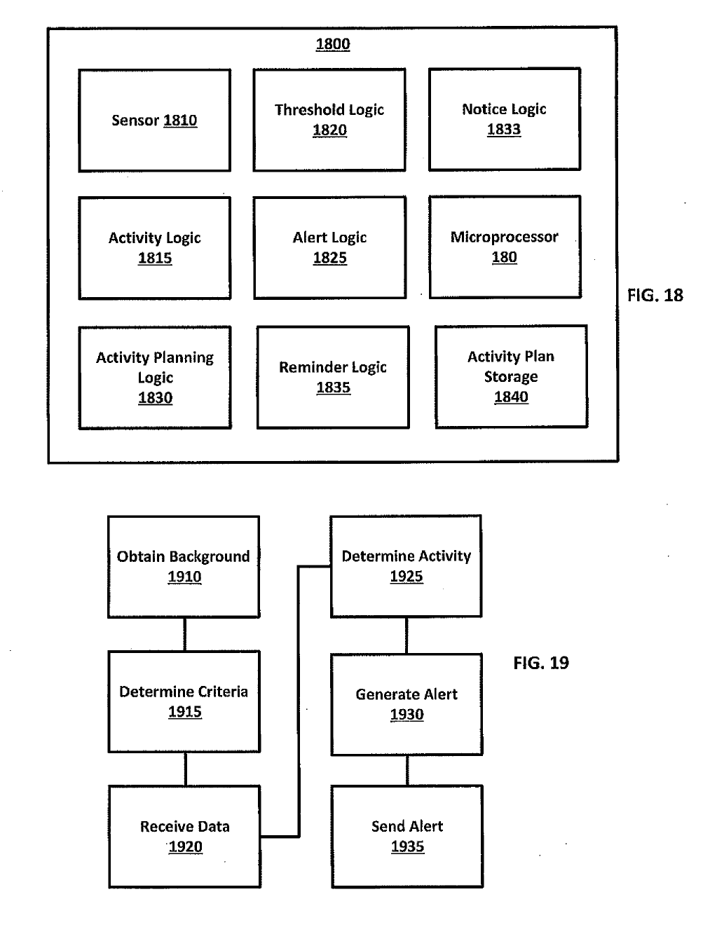

FIG. 18 illustrates a Vigilance System 1800, according to various embodiments of the invention. Vigilance System 1800 is optionally an embodiment of Monitoring System 100 and/or Activity Monitoring System 700. Vigilance System 1800 may be configured to perform Pharmacovigilance and/or other types of medical vigilance, e.g., monitoring a patient following a trauma. Vigilance System 1800 is optionally configured to detect adverse drug reactions. The drugs may include pharmaceuticals received over-the-counter or prescribed by a medical care provider, e.g., a doctor or psychiatrist. The adverse reactions can include depression, mania, bipolar disorder, schizophrenic symptoms, sleep disorders, kidney malfunction, stroke, dizziness, balance, and/or the like. The drugs may include those in clinical trial or those having received government approval.

Vigilance System 1800 is optionally configured to monitor physiological state and/or activity levels of a patient. For example, a monitored physiological state can include pulse rate, blood pressure, body position, abdominal sounds, heart and brain electrical signals, temperature, movement, breathing pattern, sleep state, shaking, balance, gait, glucose level, and/or the like. A wearable device may be configured to detect position of a specific limb.

Vigilance System 1800 includes one or more Sensors 1810 configured to detect the activity of a patient. Sensors 1810 may be configured to detect location, movement, temperature, blood pressure, sounds, electrical signals, pulse, cardiac activity, physiological state, and/or the like. Sensors 1810 may include any of the embodiments of Motion Sensor 140 and/or Sensors 715 discussed herein. In some embodiments, Vigilance System 1800 includes an interface configured to receive data from third party sensors. For example, from sensors of a home security system, smartphone, tablet computer, wearable, IoT device, and/or a medical device.

Sensors 1810 may be placed on or within a patient's body. For example, a temperature sensor may be placed in underwear or a bra. Such clothing may include one or more pockets for holding a wired or wireless temperature sensor. Pockets can be placed at the front or back of underpants, or such that the sensor is positioned between a patient's legs. Pockets may be placed in the front and/or back of a bra.

Vigilance System 1800 further includes Activity Logic 1815. Activity Logic 1815 is configured to determine an activity level of a patient based on the detected movement and/or detected location as determined using Sensors 1810 and/or patient input. The activity level may be represented as a statistical function, e.g., average, median, derivative, etc. The activity level may be over a specific time period. In some embodiments, Activity Logic 1815 is configured to receive manual input from a patient or caregiver and to determine activity levels based on this input. The input may be received via a user interface of a computing device, e.g., Monitored Device 110A.

Activity Logic 1815 optionally includes a machine learning system configured to derive activity of the user from sensor data. Activity Logic 1815 can include embodiments of Activity Logic 135 and/or Activity Analysis Logic 730. Embodiments of pharmacovigilance systems and methods provide activity logic configured to calculate a statistical function of the patient's movement.

Vigilance System 1800 optionally further includes Threshold Logic 1820 configured to set one or more criteria for generating an alert. Threshold Logic 1820 can include embodiments of Threshold Logic 750. The criteria for setting an alert can be set based on, for example, an identity of a pharmaceutical prescribed to a patient, an identity of a pharmaceutical suggested to the patient, a treatment offered to the patient, a patient's medical history, a past activity level of the patient, known pharmaceutical side effects, input from a caregiver, doctor suggestions, trauma experienced by the patient, and/or the like. The criteria set can include thresholds for more than one characteristic of activity. In certain embodiments for pharmacovigilance systems and methods, the threshold logic is configured to set a threshold above or below which an activity level is unexpected, in response to the patient receiving a pharmaceutical selected from the group consisting of an anti-depressant, an anti-psychotic, levodopa, Interferon-alpha, corticosteroids and anabolic-androgenic steroids, dopaminergic anti-Parkinsonian drugs, thyroxine, Digitalis/digoxin, Antiepileptic drugs, iproniazid, isoniazid, sympathomimetic drugs, chloroquine, baclofen, alprazolam, captopril, amphetamine and phencyclidine.

In some embodiments, Threshold Logic 1820 is configured to set a threshold that is at least 1.5, 2 or 3 standard deviations from a historic activity level. If the patient's activity exceeds an upper threshold above the historic activity level, then mania may be indicated. If the patient's activity is less than a lower threshold, then depression and/or other conditions may be indicated. In some cases, an adverse drug reaction can include swings between unusually high and unusually low activity levels, e.g., a bipolar reaction. Different levels of surveillance may be required for different pharmaceuticals. For example, some drugs may require merely vigilance using smartphone sensors, while other drugs may require vigilance using a home security system or a wearable device.

Threshold Logic 1820 is optionally configured to receive: approval from a medical provider for a threshold, prescription (dosage, drug) information, over-the counter pharmaceutical consumption information, drug side effect information, expected activity for a cohort of a patient, the patient's medical history, characterization of trauma experienced by the patient, a suggested threshold from a medical provider, the patient's activity history, and/or the like. Any of this information may be used in determining thresholds. Thresholds may be sent in response to combinations of pharmaceuticals and/or trauma. Thresholds may be set in response to treatments for a patient other than pharmaceuticals. Threshold Logic 1820 optionally includes a machine learning system configured to distinguish between expected and unexpected activity levels for a patient.

Vigilance System 1800 further includes Alert Logic 1825 configured to generate an alert when the activity levels of a patient, as determined by Activity Logic 1815, satisfies the criteria for generating the alert, e.g., when the determined activity levels deviate from expected activity levels or crosses a threshold. Alert Logic 1825 may include embodiments of Alert Logic 755 and/or Alert Logic 186. The generated alert can include a summary of a patient's activity, reasons why the alert was generated, a graphical representation of a user's activity, a listing of treatments received by the patient, pharmaceuticals received by the patient, and/or the like. In various embodiments, an alert is communicated as a text message, e-mail, and/or via a medical information portal. The alert may be communicated to a supporter in the patient's personal network, e.g., family member or close friend. The alert may also be communicated to a care provider such as a pharmacist, homecare provider, nurse, psychologist, psychiatrist, and/or doctor.

The generation of an alert by Alert Logic 1825 can be based on one characteristic of the patient's activity or a combination of different characteristics. For example, an alert may be generated in response to the patient being awake for over 24 hours more than once in a week and an increase in credit card usage. The generation of an alert by Alert Logic 1825 can be in response to patterns detected in a patient's activity. For example, periods of high activity alternating with periods of low activity can be indicative of a bipolar condition. Because thresholds are optionally set as a function of pharmaceuticals taken or to be taken by the patient, the generation of an alert by Alert Logic 1825 can be dependent on these same factors. Embodiments of Alert Logic 1825 for pharmacovigilance systems and methods includes determining one or more criteria for generating an alert. These criteria provide at least one of a patient identity or dosage of a pharmaceutical provided to the patient. Embodiments include Alert Logic 1825 that is configured by a machine learning system to determine when a patient's determined activity level satisfies the patient's criteria.

In an illustrative example, a patient having had abdominal surgery should be active, but not too active. A woman having recently given birth can be monitored for fever, depression, and/or bleeding. Alert Logic 1825 may be configured to generate an alert if the woman experiences an unexpected rise in temperature that can be indicative of an infection. A person having had a stroke may be monitored for balance and gait. There are many such conditions that can be monitored for specific trauma.

Alert Logic 1825 is optionally configured to summarize user activity data in order to protect privacy of a patient. For example, Alert Logic 1825 may work in conjunction with Filter Logic 150, configured to remove precise location information from an alert. Further, medical data such as a medical history of the patient may be removed from alerts sent to non-medical personal.

Vigilance System 1800 further includes Notice Logic 1833. Notice Logic 1833 is configured to send an alert generated by Alert Logic 1825. The alert may be sent via e-mail, text message, audio call, medical service portal, and/or other communication system. Notice Logic 1833 optionally includes embodiments of Alert Logic 755. Notice Logic 1833 is optionally configured to use I/O 130 to send alerts.

Vigilance System 1800 further includes embodiments of Microprocessor 180 configured to execute the other elements of Vigilance System 1800. For example, Microprocessor 180 may be configured to execute Activity Logic 1815, Threshold Logic 1820 and/or Alert Logic 1825.

In some embodiments, Vigilance System 1800 further includes Activity Planning Logic 1830. Activity Planning Logic 1830 is configured for determining or selecting an activity plan based, for example, on a trauma experienced by a patient. This determination is optionally automatic, i.e., performed without requiring additional human intervention. The trauma experienced by a patient may be indicated in data entered by a caregiver. For example, a doctor or nurse may enter a name of a procedure, procedure code, billing code, and/or the like. Specifically, a medical care provider may enter a code for a knee surgery and as a result Activity Planning Logic 1830 will choose an activity plan matching post-surgical guideline for this trauma and appropriate for the patient. This activity plan may indicate both a minimum and maximum level of activity. The chosen activity plan is optionally retrieved from an Activity Plan Storage 1840. As used herein, the term “storage” is used to refer to digital memory such as DRAM, RAM, SRAM, optical memory, magnetic memory, electronic medical records, and/or the like. Storage optionally includes a circuit comprising logic gates and control lines. Activity Plan Storage 1840 may be distributed among multiple devices. Activity Planning Logic 1830 optionally includes embodiments of Data Input 720.

An activity plan can include a range of expected activity such as an amount of walking, an amount of time standing, several bathroom visits, an amount of movement, a pulse rate range, a body temperature range, exercise, and/or the like. Further, the activity plan can include a range of expected activity, wherein activity outside of this range would be indicative of a mental health issue (such as depression or mania) and/or other medical issue (such as being drunk or signs of a stroke).

Activity Planning Logic 1830 is optionally further configured to select an activity based on a medical history of a patient. For example, an 80-year-old woman with a history of cardiac issues and having just received a hernia operation will likely receive a different activity plan than a 20-year-old male diabetic having just had a similar operation. A patient with a history of depression or other mental disorder is more likely to have an activity plan that would watch for mental health conditions relative to a patient without such a history. A patient taking a medication with known to cause specific side effects may receive an activity plan configured to detect these side effects. In this case, Activity Planning Logic 1830 may be configured to receive the name and/or dosage of a medication and automatically generate an activity plan that monitors for known side effects of the medication. A patient having a history of health problems may receive an activity plan configured to detect recurrence of these problems. For example, an activity plan may be configured to detect that a patient is drunk or taking drugs based on their movement or gait, that a patient is having an adverse drug reaction, that a patient is experiencing an allergic reaction (e.g., to a food, insect and/or drug), that a patient is hyper or hypo thyroid, that patient is hyper or hypo glycemic, and/or the like.

Typically, Activity Planning Logic 1830 is configured for a caregiver to modify characteristics of an activity plan via a user interface. For example, a caregiver may adjust a postpartum plan based on difficulties during delivery of a baby, or mental state of the mother. Specifically, the caregiver may reduce a threshold that would detect postpartum depression for a mother having difficulty breast feeding, a difficult birth, and/or a premature birth.

In some embodiments, Activity Planning Logic 1830 is configured to request information from a caregiver via a user interface. For example, following a birth, the caregiver may be presented with a series of questions meant to identify factors that would suggest an activity plan other than a default for the birth. Such questions could include, for example, the length of the labor, weight and size of the baby, number of babies, drugs required, damage to the birth canal, and/or the like.

In some embodiments, Vigilance System 1800 further includes Reminder Logic 1835. Reminder Logic 1835 is configured to remind a patient (e.g., user of Monitored Device 110A) to engage and/or not engage in an activity. For example, Reminder Logic 1835 may be configured to remind a new mother to get out of the house or get more sleep. Reminder Logic 1835 may be configured to remind a patient with a broken arm to exercise the arm a moderate amount. Reminder Logic 1835 may be configured to remind a kidney patient to drink more fluids or to take medications. Reminder Logic 1835 may be configured to remind a surgery patient not to go dancing a week after surgery. Reminder Logic 1835 is optionally configured to display a reminder on Display 125 of Monitored Device 110A.

FIG. 19 illustrates methods of providing pharmacovigilance, according to various embodiments of the invention. These methods are optionally performed using Vigilance System 1800. They may be applied to treatment and monitoring of patients to reduce the medical consequences of adverse pharmaceutical reactions, and/or to detect post-surgical (or other medical procedure) complications. Further they may be used in the testing and/or development of pharmaceuticals. For example, Vigilance System 1800 and the methods illustrated in FIG. 19 may be used in clinical trials. In this application, changes in activity level can be used to adjust dosages, change drug combinations, and/or to identify in real-time those patients benefitting and not-benefitting from the pharmaceutical under trial. Specifically, Vigilance System 1800 may be used for pharmacovigilance during a clinical trial. Those trial participants that show adverse reaction to the treatment (pharmaceutical or physical treatment) on trial may be removed from the trial or may receive a modified treatment according to the trial protocol. The same pharmacovigilance is then optionally used during approved use of the treatment in the general population.

In an optional Obtain Background Step 1910 a background activity level for a patient is obtained. The background activity level may be obtained using Sensors 1810 and Activity Logic 1815, and/or may be based on characteristics of the patent such as their medical history, age, gender, education, employment, residence, etc. The background activity level may be obtained using Monitored Device 110A and/or Activity Monitoring System 700. The background activity level may be obtained using any of the methods illustrated by FIGS. 3-6, 9-11 or 13.

In a Determine Criteria Step 1915, one or more criteria for generating an alert are determined. This determination is based at least in part on a medical treatment to be (or having been) received by the patient, e.g., based on an identity and/or dosage of a pharmaceutical provided and/or to be provided to the patient and/or a surgery/procedure experienced by the patient. The criteria may further be based on a medical history of the patient. In some embodiments, a medical caregiver can manually adjust criteria, e.g., raise or lower a specific threshold for a characteristic of the patient's activity. For example, a doctor may adjust a threshold for a wake/sleep pattern of a patient if the doctor is particularly concerned about the patient's reaction to a pharmaceutical that is known to cause sleep problems. Determine Criteria Step 1915 is optionally performed using Threshold Logic 1820.

In a Receive Data Step 1920 data regarding the patient's activity is received from Sensors 1810. This data may represent movement (or lack thereof) of the patient, location of the patient, specific activities pf the patient (e.g., carrying a smartphone, going to the bathroom, opening a refrigerator, driving, spending money, etc.), and/or physiological states of the patient (e.g., blood pressure, pulse rate, temperature, electrical signals, body position, abdominal sounds, breathing pattern, sleep pattern, and/or the like).

In a Determine Activity Step 1925, one or more characteristics of the patient's activity level are determined using the data received in Receive Data Step 1920. Determine Activity Step 1925 is optionally performed using Activity Logic 1815. Determine Activity Step 1925 is optionally performed using Machine Learning System 745 and/or Rule Logic 747.

In a Generate Alert Step 1930, an alert is generated in response to the one or more characteristics of the patient's activity level meeting the criteria set in Determine Criteria Step 1915. Generate Alert Step 1930 is optionally performed using Alert Logic 1825.

In a Send Alert Step 1935 the alert generated in Generate Alert Step 1930 is sent to a medical provider of the patient and/or to other supporters of the patient. For example, the alert may be sent to both a doctor and a child of an elderly patient.

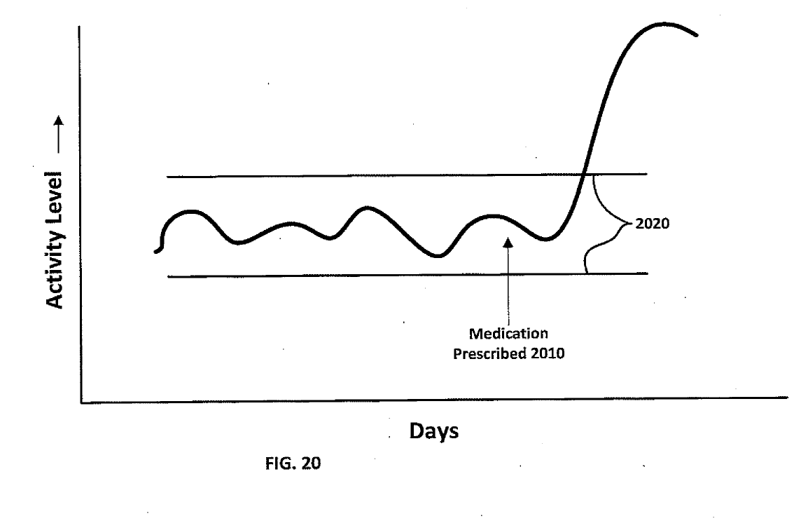

FIG. 20 illustrates a patient's activity levels over time, according to various embodiments of the invention. Shown is a 3-day moving average illustrating how much the patient's smartphone was moved during the relevant period. The moving average averages the daily variations between daytime and nighttime activity. At a Time 2010 indicated, the patient receives a medication (pharmaceutical). Thresholds for the illustrated characteristic (3-day moving average of phone movement) of the patient's activity are represented by dashed Lines 2020. In the illustrated embodiments, the thresholds are set based on at least an identity of the prescribed pharmaceutical and a historical activity of the user as measured over 2-4 weeks. The thresholds are optionally set at a predetermined number of standard deviations from a mean historical activity, in order to control a false positive rate.

Sometime after Time 2010 the activity level of the patient is seen to increase well above the upper threshold (upper Line 2020). This event typically results in generation and sending of an alert as illustrated in FIG. 19. The alert may include the graph shown in FIG. 20 and may also point out that the change in activity may be indicative (but not necessarily diagnostic) of a manic episode. The alert may suggest that a care provider evaluate the patient to determine if an adverse reaction to the prescribed medication is occurring.

In some embodiments, changes in activity level such as those illustrated in FIG. 20 would automatically prevent renewal of a prescription. Alternatively, for a patient whose activity level is already abnormally low, e.g., do to depression, a doctor may adjust the upper threshold illustrated in FIG. 20 up, such that the patient's activity may move to a more normal (healthy) range for that patient without triggering an alert. Specifically, if the historical activity level represents an unhealthy or undesirable condition, then Threshold Logic 1820 and/or a medical provider may set thresholds to include a preferred healthy range. Once this range is reached by the patient, a drop-in activity level back down to the unhealthy condition (the prior condition) may be grounds for an alert. In this case, the lower threshold is automatically adjusted up as the patient improves. In some embodiments, an alert includes a visual representation of the activity of the patient. Threshold Logic 1820 is configured to set a threshold representing a boundary between expected and unexpected levels of activity.

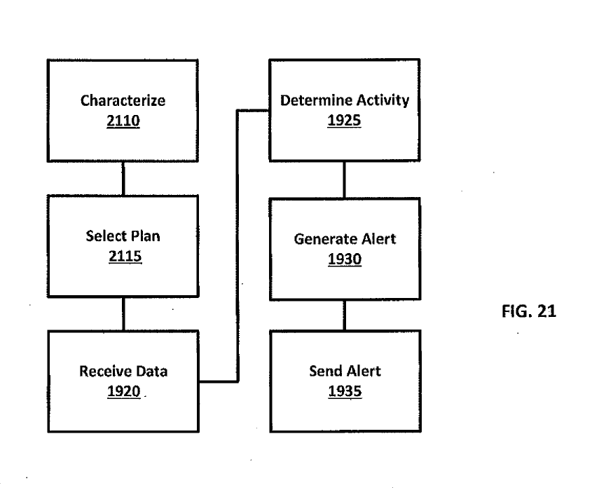

FIG. 21 illustrates a method of monitoring a patient, according to various embodiments of the invention.

In a Characterize Step 2110, a trauma experienced by a patient is characterized. As noted elsewhere herein, the characterization can include a medical procedure, a medical code, a detailed description of an injury, drugs taken, and/or the like. In some examples, a default characterization, e.g., hip replacement, is further detailed by a medical care provider. The characterization may change over time. For example, a patient having had a blood clot removed from her leg, may have the characteristics of their trauma updated when a success of the operation and after effects are determined. The amount of clearing of the clot and remaining scare tissue may be included as characteristics of the trauma.

In a Select Plan Step 2115, an activity plan for the patient is selected based on the characterization of the trauma. The selection is optionally made automatically. In some embodiments an automatically selected activity plan is manually modified by a caregiver. Characterize Step 2110 and Select Plan Step 2115 are optionally performed using Activity Planning Logic 1830.

Steps 1920 through 1935 are then performed as described elsewhere herein. In Generate Alert Step 1930 the alert is generated based on a comparison between the activity determined in Determine Activity Step 1925 and the activity plan selected in Select Plan Step 2115. In Send Alert Step 1935 the alert may be sent to a caregiver that characterized the trauma experienced by the patient. An embodiment of the Alert Step The embodiments discussed herein are illustrative of the present invention. As these embodiments of the present invention are described with reference to illustrations, various modifications or adaptations of the methods and or specific structures described may become apparent to those skilled in the art. All such modifications, adaptations, or variations that rely upon the teachings of the present invention, and through which these teachings have advanced the art, are within the spirit and scope of the present invention. Hence, these descriptions and drawings should not be considered in a limiting sense, as it is understood that the present invention is in no way limited to only the embodiments illustrated.

Computing systems referred to herein can comprise an integrated circuit, a microprocessor, a personal computer, a server, a distributed computing system, a communication device, a network device, or the like, and various combinations of the same. A computing system may also comprise volatile and/or non-volatile memory such as random access memory (RAM), dynamic random access memory (DRAM), static random access memory (SRAM), magnetic media, optical media, nano-media, a hard drive, a compact disk, a digital versatile disc (DVD), and/or other devices configured for storing analog or digital information, such as in a database. The “logic” discussed herein consists of hardware, firmware, and/or software stored on a non-transient computer readable medium, or combinations thereof. A computer-readable medium, as used herein, expressly excludes paper. Computer-implemented steps of the methods noted herein can comprise a set of instructions stored on a computer -readable medium that when executed cause the computing system to perform the steps. A computing system programmed to perform functions pursuant to instructions from program software is a special purpose computing system for performing those particular functions. Data that is manipulated by a special purpose computing system while performing those particular functions is at least electronically saved in buffers of the computing system, physically changing the special purpose computing system from one state to the next with each change to the stored data.

The logic discussed herein includes hardware, firmware and/or software stored on a computer readable medium. This logic may be implemented in an electronic device, e.g., circuit, to produce a special purpose computing system and to provide electronic medical records.

What is claimed is:

1. A pharmacovigilance system comprising:
A) a sensor configured to detect movement or location of a patient;
B) activity logic configured to determine an activity level of the patient based on the detected movement or location of the patient;
C) threshold logic configured to set one or more criteria for generating an alert, based on an identity of a pharmaceutical prescribed to the patient;
D) alert logic configured to generate an alert when the determined activity level of the patient based on the detected movement or location of the patient satisfies the one or more criteria for generating the alert;
E) notice logic configured to send the alert based on (i) an identity of a pharmaceutical prescribed to the patient and/or (ii) when the determined activity level of the patient based on the detected movement or location of the patient to a caregiver of the patient; and
D) a microprocessor configured to execute at least the threshold logic.

2. The pharmacovigilance system of claim 1, wherein the sensor is disposed on a smartphone.

3. The pharmacovigilance system of claim 1, wherein the sensor is one of a plurality of sensors configured to detect the movement or location, the plurality of sensors being disposed in different devices.

4. The pharmacovigilance system of claim 1, wherein the activity logic is distributed among several devices.

5. The pharmacovigilance system of claim 1, wherein the activity logic is configured to be executed on a mobile device.

6. The pharmacovigilance system of claim 1, wherein the activity logic is further configured to filter location data of the patient.

7. The pharmacovigilance system of claim 1, wherein the activity logic is configured to calculate a statistical function of the movement.

8. The pharmacovigilance system of claim 1, wherein the threshold logic is configured to set a threshold representing a boundary between expected and unexpected levels of activity of the patient based on the detected movement or location of the patient.

9. The pharmacovigilance system of claim 8, wherein the threshold logic is configured to set a threshold above or below which the activity level of the patient based on the detected movement or location of the patient is unexpected, in response to the patient receiving a pharmaceutical selected from a group consisting of an anti-depressant, an anti-psychotic, levodopa, Interferon-alpha, corticosteroids and anabolic-androgenic steroids, dopaminergic anti-Parkinsonian drugs, thyroxine, Digitalis/digoxin, Antiepileptic drugs, iproniazid, isoniazid, sympathomimetic drugs, chloroquine, baclofen, alprazolam, captopril, amphetamine and phencyclidine.

10. The pharmacovigilance system of claim 9, wherein the criteria set by the threshold logic comprises criteria for several different measures of patient activity.

11. The pharmacovigilance system of claim 9, wherein the criteria set by the threshold logic comprises criteria based on an activity history for the patient.

12. The pharmacovigilance system of claim 1, wherein the alert comprises a visual representation of the activity of the patient.

13. The pharmacovigilance system of claim 1, wherein the notice logic is configured to send the alert in an e-mail or a medical service portal.

14. The pharmacovigilance system of claim 1, wherein the threshold logic is configured to receive approval of the criteria from a medical caregiver.

15. The pharmacovigilance system of claim 1, wherein the threshold logic is configured to set the criteria using a machine learning system.

16. The pharmacovigilance system of claim 1, wherein the alert logic is configured to use a machine learning system to determine when the determined activity level satisfies the criteria.

17. The pharmacovigilance system of claim 1, wherein the activity logic is configured to store the activity level in an electronic medical record.

18. A method of providing pharmacovigilance, the method comprising:
A) obtaining a background activity level for a patient;
B) determining one or more criteria for generating an alert, the one or more criteria comprising at least one of a patient identity or dosage of a pharmaceutical provided to the patient;
C) receiving sensor data representative of movement of the patient;
D) determining an activity level for the patient based on the sensor data;
E) generating an alert responsive to the activity level meeting the criteria for generating an alert; and
F) sending the alert to a medical provider of the patient.

19. The method of providing pharmacovigilance of claim 18, further providing a smartphone as part of obtaining the background activity level for the patient.

20. The method of providing pharmacovigilance of claim 18, further providing a plurality of sensors configured to detect the movement or location, the plurality of sensors being disposed in different devices as part of obtaining the background activity level for the patient.

21. The method of providing pharmacovigilance of claim 18, further providing activity logic distributed among several devices as part of obtaining the background activity level for the patient.

22. The method of providing pharmacovigilance of claim 21, further providing activity logic configured to be executed on a mobile device.

23. The method of providing pharmacovigilance of claim 21, further providing activity logic configured to filter location data of the patient.

24. The method of providing pharmacovigilance of claim 21, further providing activity logic configured to calculate a statistical function of the patient's movement.

25. The method of providing pharmacovigilance of claim 18, further providing threshold logic configured to set a threshold representing a boundary between expected and unexpected levels of patient activity.

26. The method of providing pharmacovigilance of claim 25, further providing threshold logic configured to set a threshold above or below which an activity level is unexpected, in response to the patient receiving a pharmaceutical selected from a group consisting of an anti-depressant, an anti-psychotic, levodopa, Interferon-alpha, corticosteroids and anabolic-androgenic steroids, dopaminergic anti-Parkinsonian drugs, thyroxine, Digitalis/digoxin, Antiepileptic drugs, iproniazid, isoniazid, sympathomimetic drugs, chloroquine, baclofen, alprazolam, captopril, amphetamine and phencyclidine.

27. The method of providing pharmacovigilance of claim 25, further providing threshold logic comprising criteria for several different measures of patient activity.

28. The method of providing pharmacovigilance of claim 25, further providing threshold logic comprising criteria based on an activity history for the patient.

29. The method of providing pharmacovigilance of claim 18, further generating an alert comprising a visual representation of the activity of the patient.

30. The method of providing pharmacovigilance of claim 18, further generating an alert comprising communication by an e-mail or a medical service portal.

31. The method of providing pharmacovigilance of claim 18, further providing approval of the criteria from a medical caregiver.

32. The method of providing pharmacovigilance of claim 18, further providing threshold logic configured to set the criteria using a machine learning system.

33. The method of providing pharmacovigilance of claim 18, further providing alert logic configured by a machine learning system to determine when the patient's determined activity level satisfies the patient's criteria.

34. The method of providing pharmacovigilance of claim 18, further storing the patient's activity level in an electronic medical record.
